(19) United States
(12) Patent Application Publication
(10) Pub. No.: US 2023/0201393 A1
(43) Pub. Date: Jun. 29, 2023

(54) DISINFECTION SYSTEMS AND METHODS USING POLYMER COMPOSITIONS THAT FORM CHLORINE DIOXIDE GAS

(71) Applicant: CSP TECHNOLOGIES, INC., Auburn, AL (US)

(72) Inventors: John Belfance, Phenix, AL (US); Jason Pratt, Auburn, AL (US)

(21) Appl. No.: 17/906,900

(22) PCT Filed: Mar. 22, 2021
(86) PCT No.: PCT/US21/70296

§ 371 Date: Sep. 21, 2022

Related U.S. Application Data
(60) Provisional application No. 63/023,798, filed on May 12, 2020.
(60) Provisional application No. 62/993,047, filed on Mar. 22, 2020.
(60) Provisional application No. 63/004,483, filed on Apr. 2, 2020.

(51) Int. Cl. A61L 2/20

(52) U.S. Cl. CPC A61L 2/20; A61L 2101/06

(57) ABSTRACT

Disclosed methods and systems for providing safe, conveniently onsite, quick and effective ways to deliver antimicrobial and antiviral treatment to objects, which include personal protective equipment such as medical N95 type masks and other medical tools and devices, as well as other objects such as cosmetics, toys, kitchen wares, electronics and myriad of others. Chlorine dioxide gas autoclaves, room and car decontaminants and deodorizers, air filters and pest control devices are also disclosed.

### CROSS-REFERENCE TO RELATED APPLICATIONS

This application claims priority to U.S. Provisional Patent Application No. 62/993,047, entitled “USE OF CIO2 RELEASING POLYMER FILM FOR STERILIZING HOSPITAL MASKS AND OTHER ITEMS”, filed on Mar. 22, 2020; U.S. Provisional Patent Application No. 63/004,483, entitled “SYSTEM FOR DISINFECTING PERSONAL PROTECTIVE EQUIPMENT FOR REUSE UTILIZING CHLORINE DIOXIDE GAS RELEASING POLYMERS”, filed on Apr. 2, 2020; and U.S. Provisional Patent Application No. 63/023,798, entitled “DISINFECTION SYSTEMS AND METHODS UTILIZING POLYMER COMPOSITIONS THAT FORM CHLORINE DIOXIDE GAS”, filed on May 12, 2020, the contents of each of which are incorporated herein by reference in their entirety.

### FIELD OF THE INVENTION

This invention relates to a system and method for disinfection of objects, specifically by use of polymer compositions having antimicrobial properties that release chlorine dioxide gas. Chlorine dioxide gas functions in accordance with the invention as an antimicrobial agent to inhibit pathogens. The system herein will have application in the reduction, inhibition and elimination of viral, bacterial, fungal and other microbial proliferation or infection. Of particular use, the system herein may be used for the disinfection of medical devices, including protective personal equipment such as face masks. In this way, the masks may be disinfected and reused multiple times, enhancing safety to healthcare professionals and patients and addressing limited supply of such items. The disinfection of any other myriad of objects is disclosed, including cell phones, cosmetics, kitchen wares, toys, eye glasses, mail, currency and others. Additional applications of the invention include room and car sanitizers, deodorizers, air filters, and pest control devices.

### BACKGROUND OF THE INVENTION

The global COVID-19 pandemic resulted in an acute shortage of personal protective equipment, specifically N95 respirator masks necessary to protect health professionals from contracting the SARS-Cov-2 (Covid-19) coronavirus pathogen. Healthcare workers sometimes use and repeatedly reuse single use personal protective equipment (PPE) without an ability for decontamination of these devices in a safe and effective manner. The Unites States Centers for Disease Control and Prevention (CDC) recognized the decontamination and re-use of N95 masks as a crisis capacity strategy.

Medical masks are needed and used in the medical arena in order to attempt to prevent or minimize contagion by and between medical workers and patients and the public in general. Viral infections are common and are of special concern with the impact on the world of the Covid-19 coronavirus. Viruses are small infectious agents that replicate only inside living cells of an organism. Viruses are not able to reproduce if not in a living organism. While not inside an infected cell or in the process of infecting a living cell, a virus exists in the form of an independent particle, a virion. Virions consist of three parts: genetic material, a protein coat, and an outside envelope of lipids. Viruses can vary in shape and size. Common viruses include the common cold, influenza, hepatitis, SARS (including coronavirus), measles, rotavirus, and many others. Viruses have a variety of transmission mechanisms and rates. Each virus also has a specific transmission spread: some spread via coughing and sneezing, while others spread via sexual contact, or fecal-oral routes. Some viruses like coronavirus Covid-19 are particularly infectious when airborne. Although some viruses are combatable though current vaccinations or antiviral agents, many viruses do not yet have a known treatment, therefore it is particularly critical to minimize their spread and contagion.

Numerous medical masks have been developed. Such devices vary in their ability to prevent contagion between the health professionals and patients or between the general public wearing such masks. In the United States, one such mask is the N95 mask. An N95 mask, also called a “respirator”, is a mask that is worn over the face to prevent the inhalation of airborne particles. The N95 designation means that the mask will filter at least 95% of particles of 0.3 microns in size or larger. During the Covid-19 pandemic, an insufficient number of masks have been available to both healthcare professionals and to the public needed in order to prevent contagion and infection by healthcare workers, patients and the general population. An alternative to use of new masks is the disinfection and/or sterilization and/or decontaminate and reuse of existing masks, when performed in a safe and effective manner.

Various methods exist to sterilize personal protective equipment including ionizing radiation, sterilization with ethylene oxide (EtO), microwave-generated steam (MGS), ultraviolet germicidal irradiation (UVGI), moist heat, bleach, liquid hydrogen peroxide (LHP), and hydrogen peroxide gas plasma (HPGP). However, these decontamination procedures either require specialized materials, equipment, or facilities, or may be unsafe unless properly performed by a professional with specialized training. Further, some of these processes reduce the filtration and performance of the personal protective equipment being sterilized. An effective, safe and easy to use method of disinfection/sterilization/decontamination is needed.

The Battelle Critical Care Decontamination System™ (from Battelle Memorial Institute of Columbus, Ohio, USA) was authorized and became available in March 2020 in response to the Covid-19 pandemic. The Battelle decontamination system purported successful testing on decontaminated N95 respirators demonstrating acceptable performance through 20 decontamination cycles for sporicidal activity, viricidal activity, filtration efficiency, breathability, form fit testing, and strap integrity testing, per authorized respirator. The Battelle system is a self-contained decontamination device that uses vapor phase hydrogen peroxide (VPHP) for decontamination of compatible N95 or N95-equivalent respirators that are contaminated or potentially contaminated with SARS-CoV-2. The Battelle process is incompatible with N95 or N95-equivalent respirators that contain cellulose-based materials. Each decontamination cycle in the Battelle decontamination system consists of injecting VPHP into the decontamination chamber until achieving a saturated atmosphere indicated by micro condensation; maintaining the VPHP exposure for a 150-minute dwell time; and allowing the VPHP to off gas to a level of 1 ppm prior to post decontamination processing. A minimum of five calibrated chemical indicators are dispersed throughout the system to indicate a successful decontamination cycle. This decontamination system enables the reuse of compatible N95 or N95-equivalent respirators that would otherwise be disposed after a single use. However, the Battelle system was made available under the Emergency Use Authorization guidelines of the U.S. Food and Drug Administration (FDA) and did not undergo stringent safety and efficacy review as an FDA-approved or cleared device. Thus, a proven safe and effective means for decontamination of N95 and N95-equivalent masks continues to be needed. Moreover, the Battelle system requires complicated and expensive specialized equipment to operate.

The U.S. FDA consulted with subject matter experts on the public health needs for a decontamination system of N95 masks to prevent the spread of the COVID-19 pathogen. The FDA concluded that there currently exists a public health need for such devices due to a lack of an adequate available alternative system for reducing the bioburden on N95 respirators during the public health emergency.

Due to lack of availability of medical masks and/or the lack of a readily available and accessible system to decontaminate masks easily, in an attempt to prevent infection and the spread of infection, as well as a desire to have more fashionable alternatives, medical professionals and the general population have resorted to using woven and nonwoven fabrics and other easily available materials to make homemade masks. However, woven and nonwoven fabrics lack sufficient ability to trap bacteria and viruses, which are able to pass through the fabric and potentially cause infection. To increase the ability to trap bacteria and viruses, multiple layers of the nonwoven fabric must be built up which make the masks cumbersome, may cause difficulty breathing, may increase the risk of bacterial growth and proliferation (especially in the buccal cavity and lungs) and may potentially remain insufficiently effective in preventing bacterial or viral infection.

As such, a great need exists for a system to quickly, safely, and effectively disinfect medical masks and other personal protective equipment. Such a system would allow nurses, doctors, dentists, patients and the general public to reuse their masks and not resort to use of self-made or substandard commercial masks that are insufficient in preventing the spread of infection and which would continue to expose and potentially endanger the wearer and the public.

It is known that antimicrobial agents can be incorporated into medical face masks in order to attempt to control, reduce and inactive pathogenic microbes. However, after a certain amount of time of exposure to a pathogen, the masks and other personal protective equipment become contaminated and require disinfection. A safe and effective system for disinfection, sterilization or decontamination is needed so that the personal protective equipment can be reused in a safe and effective manner when needed in times when new personal protective equipment is unavailable. A further advantage of a decontamination system for personal protective equipment is the cost reduction as compared to the sourcing of new equipment, especially to hospitals, clinics and other medical facilities as healthcare institutions often have capped, constrained or limited budgets.

Chlorine dioxide (ClO₂) has been shown to be effective as an antimicrobial agent in reducing pathogens. It has also been shown to be effective against a variety of viruses. Products containing ClO₂ gas are used for agricultural, commercial, industrial, medical and residential use antibacterial application. Specifically, the gaseous effects of chlorine dioxide against Influenza A were studied by Ogata, Samp and Shibata in 2008. This team showed that 0.03 ppm of ClO₂ could have an effective 4 log kill when administered at the same time as the virus and if administered after the survival rate increased to 100% versus 30% when untreated. Ogata, N., Shibata, T. Protective effect of low-concentration chlorine dioxide gas against influenza A virus infection. Journal of General Virology, 89(1), 60-67, (2008). Harakeh gives data that shows certain types of viruses can be inactivated by as much as or more than 99.9% by 4 ppm concentration of ClO₂ after 5 minutes of exposure, including human rotavirus, coxsackievirus B5, echovirus 1, poliovirus 1, bacteriophage f2, and siamian rotovirus. Harakeh, S. The behavior of viruses on disinfection by chlorine dioxide and other disinfectants in effluent. FEMS Microbiology Letters, 44(3), 335-341, (1987). A study by Sanekata et al showed that a concentration of 1.0 ppm of ClO₂ for 180 seconds could achieve a 2 to 4 log kill on Infectious Flacherie Virus (IFV), measles, and HHV-1. Sanekata, T., Fukuda, T., Miura, T., Morino, H., Lee, C., Maeda, K., Shibata, T. Evaluation of the Antiviral Activity of Chlorine Dioxide and Sodium Hypochlorite against Feline Calicivirus, Human Influenza Virus, Measles Virus, Canine Distemper Virus, Human Herpesvirus, Human Adenovirus, Canine Adenovirus and Canine Parvovirus. Biocontrol Science, 15(2), 45-49, (2010). Simonet, Samp, and Gantzer showed that polio can also be inactivated with exposure to chlorine dioxide. Simonet, J., Gantzer, C. Degradation of the Poliovirus 1 genome by chlorine dioxide. Journal of Applied Microbiology, 100(4), 862-870, (2006).

A challenge to decontamination of personal protective equipment, and especially of medical masks and non-medical masks that can be reused, is in meeting this need in a safe and nontoxic way since the antimicrobial agent itself can also be harmful to the wearer. A system is needed that provides sufficient means for delivery of an effective amount of pathogen destroying or inactivating antimicrobial agent while ensuring that the mask is safe to the wearer after the mask has been exposed to the potentially harmful active agent. The U.S. Centers for Disease Control and Prevention (CDC) sets forth the exposure limit for chlorine dioxide gas as 0.1 ppm over an average of a 10-hour work shift, and 0.3 ppm (0.83 mg/m3) for an average of 15 minutes. Thus, a need exists for a disinfection system for masks and other personal protective equipment using chlorine dioxide that can be tailored to provide a controllable release profile for use in a mask or other medical protective gear or other medical apparatus that meets this safety criteria.

A further need exists for a disinfection system that upon use does not significantly alter or destroy the material of the personal protective equipment or alter or destroy its effectiveness in inhibiting or preventing penetration by infectious agents. An additional need exists for a disinfection system for masks and other personal protective equipment that is uncomplicated to use, with simple procedure of use and simple instructions, so that any healthcare worker, as well as an average person without healthcare or scientific training, can easily comprehend and learn to utilize the disinfection system. A yet additional need exists for a disinfection system that can be used quickly by a medical worker so that the personal protective equipment can be readily available when needed in the event that an alternative piece of personal protective equipment is not available, especially when needed in time of an emergency.

In certain embodiments, disclosed herein is a system and method for reduction of bioburden on medical masks in addition to other objects. The disclosed system offers a unique set of benefits. The system is low-cost and does not require specialized equipment, training, or servicing. Therefore, it can be easily and quickly deployed across the world. The system herein does not require transport of used N95 respirators to an off-site facility, which adds to the time and expense of the process. The system permits users to treat and keep their own N95 respirators. It is especially well-suited for smaller clinics, dental offices, urgent care centers, nursing homes, university clinics, tribal and rural healthcare facilities. In total, it provides a very practical means of supplementing CDC re-use guidelines to achieve added safety. A particular benefit of the disclosed system is that it is scalable to as many treatment units as are necessary. This scale will contribute significantly to mitigating the current shortfall in N95 respirators.

Further need exists in the field of room sanitation or sterilization. As with disinfection of medical equipment, rooms in hospitals and other medical facilities require consistent, repetitive, safe and effective sanitation procedures. Typical procedures include the employment of cleaning staff who work at night to cleanse rooms and objects within the rooms of the medical facility. However, such practices are inconsistent since they depend on the performance of individual personnel, the cleaning may be unreliable and it may potentially expose the cleaning personnel to infectious pathogens in the medical facility. A self-functioning system of disinfection is greatly needed. Furthermore, it is particularly desirable to have a system of room sanitation which can be carried out in a continuous manner while persons remain within the hospital room, and at any time of day or night. Such systems are desired not only in medical facilities, but in any environment where antimicrobial, viral, fungal or other microbial agents may be found, which includes almost any inhabited living space, such as homes, offices and commercial establishments.

### SUMMARY OF THE INVENTION

Accordingly, in one aspect, disclosed herein is a system for disinfection, decontamination, sanitation, or sterilization of personal protective equipment (PPE), including medical or personal use N95 and other masks. The system herein is also useful for the disinfection, decontamination, sanitation and/or sterilization of any object in general. The disinfection system comprises the use of polymer compositions incorporating a chlorine dioxide gas forming agent which is capable of forming and releasing chlorine dioxide gas as an active agent that functions to inhibit microbial proliferation.

In one particular embodiment, disclosed is a system where antimicrobial strips are formed using three-phase entrained polymer technology (Activ-Shield™ technology by Aptar CSP Technologies Inc., Auburn Ala., USA.) In alternate embodiments, the chloride dioxide gas forming agents used herein include chlorite salts, including alkali metal chlorites, alkaline earth metal chlorite or a transition metal chlorite. Moisture activates the metal chlorite to form chlorine dioxide gas.

The three-phase polymer provides the ability to control small molecule transport through the polymer. The pathways created by the interaction of these constituents allow for the controlled movement of chlorine dioxide gas into and out of the polymer. This has enabled the ability to engineer compounds that transmit ClO₂ gas molecules. When released into a sealed atmosphere of a package, the entrained polymer allows maintaining an optimal environment with minimal or reduced microbial count.

One method according to an optional aspect of the invention comprises the following steps: (a) placing the object to be disinfected into a container having an interior space therein, a headspace being formed of a portion of the interior space that is not occupied by the object; (b) placing into the interior space a polymer composition comprising: (i) a base polymer; (ii) a chlorine dioxide gas forming agent; and (iii) a channeling agent that forms channels though the base polymer; (c) contacting the polymer composition with moisture to form chlorine dioxide gas; and (d) enclosing the container sufficiently enough to allow the chlorine dioxide gas to accumulate in the headspace, wherein the chlorine dioxide gas disinfects the object; wherein the amount of chlorine dioxide gas on the disinfected object is substantially or completely undetectable immediately after removal of the object from the container; optionally, within 1 minute after removal; optionally within 5 minutes after removal; optionally within 10 minutes after removal; or optionally within one hour after removal; alternatively, wherein the amount of chlorine dioxide gas on the disinfected object is less than 0.01 ppm immediately after removal of the object from the container; optionally, within 1 minute after removal; optionally within 5 minutes after removal; optionally within 10 minutes after removal; alternatively, wherein the amount of chlorine dioxide gas in the ambient environment around the container is substantially or completely undetectable the entire time that the method is performed and optionally immediately after removal of the object from the container, optionally within 1 minute after removal; alternatively, wherein the amount of chlorine dioxide gas in the ambient environment around the container is less than 0.01 ppm the entire time that the method is performed and optionally immediately after removal of the object from the container, optionally, within 1 minute after removal; alternatively, wherein the amount of chlorine dioxide gas in the ambient environment around the container is considered Generally Recognized as Safe (GRAS) pursuant to Sections 201(s) and 409 of the United States Federal Food, Drug, and Cosmetic Act the entire time that the method is performed and optionally immediately after removal of the object from the container, optionally, within 1 minute after removal.

According to one preferred embodiment, a N95 mask (that has been previously used or has been exposed to microbes) is placed into a one gallon plastic sealable bag. An entrained polymer film strip according to the invention is removed from a vial (using tweezers or other means), a marker is used to draw a line (e.g., of pink ink) directly on both sides of the film strip as a chemical indicator for ClO₂ activity. The user briefly submerges the entire strip in a cup of water for one to two seconds, places the strip into the plastic bag with the N95 mask, and seals the bag. The water triggers a slow release of chlorine dioxide gas from the entrained polymer film. During the cycle, the pink line disappears, qualitatively signifying that the strip has been activated. The mask is left to be disinfected by the polymer strip in the bag for approximate 10 hours. The mask is then removed from the bag and is ready to be reused.

The amount of chlorine dioxide formed by the polymer composition is controlled by several means. In one optional embodiment, the amount of chlorine dioxide gas released into a room is 0.001 ppm to 0.1 ppm over an average of a 10-hour work shift. In an alternate optional embodiment, the amount of chlorine dioxide released is approximately 0.3 ppm over a time period of 15 minutes. Such embodiments are the ranges considered as safe for use by humans by the U.S. Center of Disease Control and Prevention (CDC), but are not limited thereto.

Optionally, in any embodiment involving disinfection of objects contaminated by microbes, the concentration of chlorine dioxide gas formed in the container effectuates a reduction of infectious viral or bacterial pathogens on the object to be disinfected, the reduction being at least a 1 log based 10 reduction in the number of such particles, optionally at least a 2 log based 10 reduction in the number of such particles, optionally at least a 3 log based 10 reduction in the number of such particles, optionally at least a 4 log based 10 reduction in the number of such particles, optionally at least a 5 log based 10 reduction in the number of such particles, optionally at least a 6 log based 10 reduction in the number of such particles, optionally at least a 7 log based 10 reduction in the number of such particles, optionally at least a 8 log based 10 reduction in the number of such particles, as compared to the initial number of such particles.

In accordance with another aspect of the present invention, the profile release rate and duration of chlorine dioxide gas formation can be designed and controlled.

It is yet an additional important component of embodiments of the invention that during the disinfection process, the amount of chlorine dioxide gas in the ambient environment around the container is substantially or completely undetectable the entire time that the method is performed and optionally immediately after removal of the object from the container, optionally within 1 minute after removal.

Optionally, colorants or color indicators are added to the polymer composition as as standalone indicators to indicate chlorine dioxide activity. Optionally, the concentration of a colorant is approximately 1% to 3%, optionally about 2% of the total weight of the polymer composition. A marker or similar gage can also be used to indicate and monitor the activity of the chlorine dioxide gas in the system.

Room decontaminants, air filters, deodorizers, and pest control devices using the chlorine dioxide gas forming polymer composition systems and methods are also disclosed.

### BRIEF DESCRIPTION OF THE DRAWINGS

The invention will be described in conjunction with the following drawings in which like reference numerals designate like elements and wherein:

FIG. 1 is a perspective schematic view of a film formed of an entrained polymer comprising a chlorine dioxide gas (ClO₂) releasing agent according an optional aspect of the invention.

FIG. 2 is a cross section taken along line 2-2 of FIG. 1, providing a schematic illustration of ClO₂ releasing agent within channels of the entrained polymer composition.

FIG. 3 is a perspective view of a flip top container that may be used to store ClO₂ releasing film to protect it from moisture in the environment, according to an optional aspect of the disclosed concept used in accordance with the system herein.

FIG. 4 is a graph showing ClO₂ gas concentration in a one gallon sealable clear plastic bag according to an exemplary embodiment of the disclosed system.

FIG. 5 is a bar graph of data measured comparing ClO₂ gas concentration as between different brands of zipper sealable plastic bags.

FIG. 6 is a line graph comparing ClO₂ gas concentration over time under certain conditions of films having different thickness, configuration or formulation according to exemplary embodiments.

FIG. 7 is a graph showing test results of a log reduction in bacterial count of various types of bacteria using an embodiment of the ClO₂ forming system according the invention.

FIG. 8 is a graph showing the best fit curve calculated for the relationship between strip equivalent dose and log reduction in bacterial count using an embodiment of the system of the invention.

FIG. 9 is a graph showing the best fit curve calculated for the relationship between strip equivalent dose and log reduction in viral count of the SARS-CoV virus using an embodiment of the system.

FIG. 10 is a graph showing peak ClO₂ gas concentration and dissipation in a one gallon plastic bag measured over 120 minutes according to an exemplary embodiment.

FIG. 11 is a photograph showing an N95 mask disposed within a one gallon resealable plastic bag for disinfection according to an exemplary embodiment of the system herein.

FIG. 12 is a graph showing ClO₂ gas concentration on masks treated with disinfection cycles according to the method herein measured immediately after their removal from sealed plastic bags.

FIG. 13 is a graph of the average headspace concentration of ClO₂ of one dosage of a film strip of an N95 respirator in a one gallon plastic bag according to an embodiment of the invention.

FIG. 14 is a graph showing the test results of a puncture study of ClO₂ concentration levels from simulated failures and controls tested according to the system of the invention.

### DETAILED DESCRIPTION OF THE INVENTION

### Definitions

As used herein, the term “antimicrobial” or “antimicrobial agent” refers to a substance that inhibits microorganisms. Classes of antimicrobials include antivirals, antibacterials, antifungals, antiparasites and other anti-pathogenic agents.

As used herein, the term “base polymer” is a polymer used according to the invention that is capable of being formed with a chlorine dioxide gas forming agent, and optionally having a gas transmission rate of a selected material that is substantially lower than, lower than or substantially equivalent to, that of a channeling agent. By way of example, such a transmission rate is a water vapor transmission rate in embodiments where the chlorine dioxide gas forming agent is activated by moisture. The primary function of the base polymer is to provide structure for the polymer entrained with chlorine dioxide gas forming agent.

As used herein, the term “channeling agent” is defined as a material that is immiscible with the base polymer and has an affinity to transport a gas phase substance at a faster rate than the base polymer alone. Optionally, a channeling agent is capable of forming channels through the entrained polymer when formed by mixing the channeling agent with the base polymer. Optionally, such channels are capable of transmitting a selected material, such as water, chlorine dioxide or others, through the entrained polymer at a faster rate than then the selected material would have in the base polymer without the channeling agent. As used herein, the term “channels” or “interconnecting channels” is defined as passages formed of the channeling agent that penetrate through the base polymer and may be interconnected with each other.

As used herein, the term “chlorine dioxide gas forming agent” refers to a compound that upon contact with moisture reacts to form chlorine dioxide, which is released in gas form.

As used herein, the terms “close”, “closed” and “closing” are used interchangeably with the terms “seal”, “sealed” and “sealing”, respectively, in reference to a container or chamber or room, as the case may be, and refer to a container or chamber or room, (respectively), being enclosed sufficiently enough to the extent that the amount of chlorine dioxide gas that is formed and remains inside the container or chamber or room, (respectively), when enclosed is on balance greater than the amount of chlorine dioxide gas that exits the container or chamber or room, (respectively), such that the chlorine dioxide gas accumulates inside the container or chamber or room, (respectively), and reaches a measurable concentration inside the container or chamber or room, (respectively.) Sealing is needed to minimize permeation of both moisture and chlorine dioxide gas through the container wall and ingress through the seal, which will also be a factor of the amount of time that the object is retained in the container. The container or chamber or room may be enclosed by any means or with any closing device appropriate thereto, as will be described.

As used herein, the terms “decontamination”, “disinfection”, “sanitization”, and “sterilization” (and “decontaminate”, “disinfect”, “sanitize”, and “sterilize” and conjugated forms thereof,) are defined herein to mean the action of contacting an object with chlorine dioxide in order to inhibit an infectious agent such as a bacteria, a virus, a fungus, a parasite or other.

The terms decontamination, disinfection, sanitization, and sterilization are often colloquially used interchangeably in general lexicon. Varying definitions are available and are provided by way of background, information and guidance in interpretation but the terms are used as defined herein. The terms have also acquired certain meanings within various fields of practice, (e.g. in chemistry, medicine, food science, and others). These terms are also are specially defined by various organizations for specific purposes, such as the U.S. Center for Disease Control (CDC), Environmental Protection Agency (EPA) and Food and Drug Administration (FDA). The definitions set forth by the CDC, EPA and FDA are provided by way of example and information only and are not intended to be limiting, unless otherwise stated in a given instance or claim. Furthermore, for purposes of clarity, the terms “decontamination”, “disinfection”, “sanitization”, “sterilization” (as well as the terms “decontaminate”, “disinfect”, “sanitize”, and “sterilize” and their conjugated forms) are used interchangeably with one another herein and each time one of these terms is used throughout the specification and claims, such term is intended to include and cover all four terms and iterations listed, unless otherwise indicated in a given instance or claim.

The U.S. CDC provides the following definitions: “Decontamination: The use of physical or chemical means to remove, inactivate, or destroy blood borne pathogens on a surface or item to the point where they are no longer capable of transmitting infectious particles and the surface or item is rendered safe for handling, use, or disposal. In health-care facilities, the term generally refers to all pathogenic organisms.” “Disinfection: Thermal or chemical destruction of pathogenic and other types of microorganisms. Disinfection is less lethal than sterilization because it destroys most recognized pathogenic microorganisms but not necessarily all microbial forms (e.g., bacterial spores).” “Sanitizer: Agent that reduces the number of bacterial contaminants to safe levels as judged by public health requirements. Commonly used with substances applied to inanimate objects. According to the protocol for the official sanitizer test, a sanitizer is a chemical that kills 99.999% of the specific test bacteria in 30 seconds under the conditions of the test.” “Sterile or Sterility: State of being free from all living microorganisms. In practice, usually described as a probability function, e.g., as the probability of a microorganism surviving sterilization being one in one million.” “Sterilization: Validated process used to render a product free of all forms of viable microorganisms. In a sterilization process, the presence of microorganisms on any individual item can be expressed in terms of probability. Although this probability can be reduced to a very low number, it can never be reduced to zero.” [C.F.R. Title 29 Section 1910.1030] (see https://www.cdc.gov/infectioncontrol/guidelines/disinfection/glos sary.html)]. As used herein, the term “sterilized” in addition to and consistent with the definition above, (meaning an object contacted with chlorine dioxide in order to inhibit a microorganism), includes that the object is free of all forms of viable microorganisms, described as a probability of a microorganism surviving sterilization being one in one million, as set forth by the U.S. CDC pursuant to the Code of Federal Regulations, Title 21, Section 110.3(o).

The U.S. EPA defines “sanitizer” as “a substance, or mixture of substances, that reduces the bacteria population in the inanimate environment by significant numbers, but does not destroy or eliminate all bacteria.” The term “disinfectant” is defined as “a substance or mixture of substances, that destroys or irreversibly inactivates bacteria, fungi, and viruses, but not necessarily bacterial spores, in the inanimate environment.” [C.F.R. Title 40, Section 158.2203.]

The U.S. FDA defines “sterilization,” in a document titled “Liquid Chemical Sterilants/High Level Disinfectants Guidance”, (https://www.cdc.gov/infectioncontrol/guidelines/disinfection/tables/table1.html), as “a validated process used to render a product free of all forms of viable microorganisms. In many cases, thermal methods, such as steam, are used to achieve sterilization. Thermal sterilization methods have been studied and characterized extensively. In addition, the survival kinetics for gas/vapor/plasma low temperature sterilization methods have also been well characterized.” “Sanitize” is defined as: “means to adequately treat food-contact surfaces by a process that is effective in destroying vegetative cells of microorganisms of public health significance, and in substantially reducing numbers of other undesirable microorganisms, but without adversely affecting the product or its safety for the consumer.” [C.F.R. Title 21, Section 110.3(o).]

As used herein, the term “headspace” refers to the portion of the interior space of a container that is not occupied by an object within the container.

As used herein, the term “infectious agent” refers to a microorganism of any species of a virus, bacteria, fungus, algae, parasite, other microbe that is capable of infecting a living organism and is capable of being modified by contact with chlorine dioxide. The infectious agent is typically but not necessarily a pathogen. The terms “infectious agent”, “microbial agent” and “pathogen” are used interchangeably herein.

As used herein, the term “inhibit” refers to the ability of chlorine dioxide to modify, hinder, restrain, prohibit, reduce, halt, inactivate, kill, stop or essentially prevent an infectious agent in its capacity to grow and/or proliferate and/or to infect another organism. All such terms are used interchangeably herein. The inhibition of antimicrobial growth may further aid in the prevention of infectious diseases caused by the virus, bacteria, fungus, algae, parasite or other microbial agents that are spread by persons touching an infected object, by airborne pathogenic transmission or by other mechanisms of transmission.

As used herein, the term “moisture” refers to and includes water (having the general chemical formula H₂O), steam (water in the form of steam), water vapor (water in its gaseous state, also commonly referred to as steam, which is typically vaporized by boiling or evaporation of water), vapor of liquid substances containing water, ambient air (ambient moisture in the environment containing water molecules or water in gas form), water molecules in a liquid other than water; as well as acetone, and/or alcohols including methanol, ethanol, propanol, butanol or ethylene glycol, as well as polar solvents, and/or combinations of any of the foregoing.

As used herein, the term “monolithic,” in “monolithic composition” is defined as a substance that is made of one essentially admixed or blended composition of materials, such that it does not itself consist of two or more discrete macroscopic layers or portions. Accordingly, a monolithic composition does not include a multi-layer composite, although a monolithic composition could form a layer of such a composite.

As used herein, the term “phase” is defined as a portion or component of a monolithic composition that is uniformly distributed throughout, to give the structure or composition its monolithic characteristics.

As used herein, the term “polymer composition” is defined as a monolithic material formed of at least a base polymer with the chlorine dioxide gas forming agent and optionally also a channeling agent distributed throughout the base polymer. A polymer composition thus includes two-phase polymers (without a channeling agent) and three phase polymers (with a channeling agent).

As used herein, the term “three phase” is defined as a monolithic composition or structure comprising three or more phases. An example of a three phase composition according to the invention is an entrained polymer formed of a base polymer, chlorine dioxide gas forming agent, and channeling agent in an amount sufficient to form channels. Optionally, a three phase composition or structure may include none or more additional compounds, (e.g., a colorant), but is nonetheless still considered “three phase” on account of the presence of the three primary functional components.

As used herein, the term “N95” mask is defined as a respirator mask that satisfies the definition of an N95 mask pursuant to regulations of the National Institute for Occupational Safety and Health of the United States (NIOSH).

### Polymer Compositions

The chlorine dioxide gas forming agent is a component of a polymer composition, preferably a three phase entrained polymer comprising the chlorine dioxide gas forming agent, a base polymer and a channeling agent. The polymer compositions herein are three phase formulations (i.e., comprising a base polymer, active agent and channeling agent). Entrained polymer compositions are described, for example, in U.S. Pat. Nos. 5,911,937, 6,080,350, 6,124,006, 6,130,263, 6,194,079, 6,214,255, 6,486,231, 7,005,459, and U.S. Patent Publication No. 2016/0039955, each of which is incorporated herein by reference as if fully set forth.

Suitable base polymers include thermoplastic polymers, including but not limited to polypropylene, polyethylene, polyisoprene, polyhydroxyalkanoates (PHAs), polylactique acid (PLA), polybutylene succinate (PBS), polyhexene, polybutadiene, polybutene, polysiloxane, polycarbonate, polyamide, ethyl vinyl acetate, ethylene-vinyl acetate (EVA) copolymer, ethylene-methacrylate copolymer, polyvinyl chloride (PVC), polystyrene, polyester, polyanhydride, polyacrylianitrile, polysulfone, polyacrylic ester, acrylic, polyurethane, polyacetal, polyvinylpyrrolidone (PVP), a copolymer, and combinations thereof.

Optionally, in any embodiment, the concentration of the base polymer within the polymer composition is in a range from 10% to 80%, optionally from 20% to 70%, optionally from 30% to 60%, optionally from 40% to 50%, optionally from 45% to 65%, optionally from 45% to 60%, optionally from 45% to 55%, optionally from 50% to 70%, optionally from 50% to 60%, optionally from 55% to 65%, optionally from 55% to 60% by weight of the total weight of the polymer composition.

The polymer compositions herein incorporate channeling agents which form channels between the surface of the polymer composition and its interior in order to transmit moisture or gas, to absorb or adsorb the moisture or gas, and to allow reaction of the moisture or gas with the chlorine dioxide gas forming agent. The channels are mainly formed of the channeling agent itself. The channeling agent used herein has a water vapor transmission rate of at least two times that of the base polymer. In other embodiments, the channeling agent has a water vapor transmission rate of at least five times that of the base polymer. In other embodiments, the channeling agent has a water vapor transmission rate of at least ten times that of the base polymer. In still other embodiments, the channeling agent may have a water vapor transmission rate of at least twenty, fifty or one hundred times that of the base polymer.

Suitable channeling agents include a polyglycol such as polyethylene glycol (PEG), ethylene-vinyl alcohol (EVOH), polyvinyl alcohol (PVOH), glycerin polyamine, polyurethane and polycarboxylic acid including polyacrylic acid or polymethacrylic acid. Alternatively, the channeling agent can be, for example, a water insoluble polymer, such as a propylene oxide polymerisate-monobutyl ether, such as polyglykol B01/240, produced by Clariant Specialty Chemicals. In other embodiments, the channeling agent could be a propylene oxide polymerisate monobutyl ether, such as polyglykol B01/20, produced by Clariant Specialty Chemicals, propylene oxide polymerisate, such as polyglykol D01/240, produced by Clariant Specialty Chemicals, ethylene vinyl acetate, nylon 6, nylon 66, or any combination of the foregoing.

Optionally, in any embodiment, the concentration of the channeling agent in the polymer composition is in a range from 1% to 25%, optionally from 2% to 15%, optionally from 5% to 20%, optionally from 8% to 15%, optionally from 10% to 20%, optionally from 10% to 15%, optionally from 10% to 12%, optionally from 5% to 15%, optionally about 7% by weight of the total weight of the polymer composition.

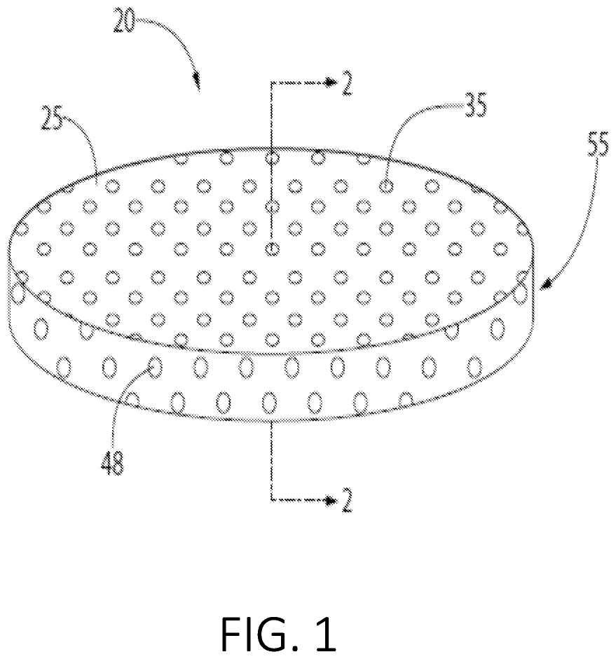

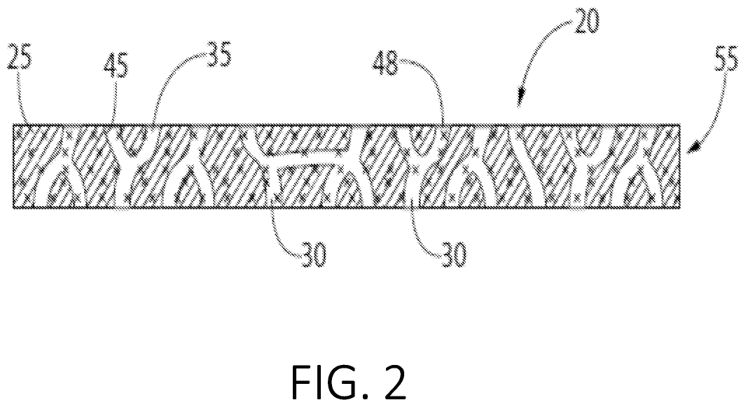

The Figures herein illustrate the entrained polymer compositions used according to the invention and various aspects and data relating to the same. FIG. 1 is a perspective schematic view of a film 55 that has been constructed from an entrained polymer 20 comprising the base polymer 25 that has been uniformly blended with the chlorine dioxide gas forming agent 30 (shown on FIG. 2) and the optional channeling agent 35. FIG. 2 is a cross-sectional view along line 2-2 of entrained polymer composition 20 of FIG. 1. The polymer composition has been solidified so that interconnecting channels 45 have formed throughout base polymer 25 to establish passages throughout the film 55. Moisture (not shown) is capable of moving from the exterior of the entrained polymer composition 20, through the channels 45, to the molecules of the chlorine dioxide gas forming agent 35, which are activated by the moisture such that chlorine dioxide gas (not shown) is formed and released from the entrained polymer composition 20. The interconnecting channels 45 facilitate transmission of the moisture and gas through the base polymer 25. The passages terminate in channel openings 48 (shown on FIG. 1) at exterior surfaces of the film 55, creating more surface area for chlorine dioxide gas and/or moisture and or other optional microbial agents to penetrate from and to the environment surrounding the polymer composition.

The polymer compositions used according to the invention herein may be prepared by any known and common manufacturing processes such as extrusion, injection molding, blow molding, thermoforming, vacuum molding, casting, continuous compounding and hot melt dispensing. In the process of manufacture, the chlorine dioxide gas forming agent is added to one or more base polymers, and optionally, one or more channeling agents, and the materials are combined and generally admixed or blended with one another to some degree. The produced combination of the base polymer mixed with the chlorine dioxide gas forming agent becomes an entrained polymer composition. The chlorine dioxide gas forming agent does not need to be distributed uniformly throughout the base polymer in order to render its antimicrobial releasing properties and embodiments may be configured accordingly. In a preferred embodiment, the chlorine dioxide gas forming agent is uniformly or essentially uniformly distributed within the base polymer such that the entrained polymer composition becomes homogeneous or essentially homogeneous. In this way, a given unit mass or volume of the composition should exhibit uniform performance characteristics under the same conditions. The chlorine dioxide gas forming agent is preferably added to the base polymer in powder form.

Various plasticizers or dispersants may be used as additives to the base polymer or polymer compositions herein to modify the plasticity or modify the viscosity of the base polymer which will affect the size of the channeling agents. Plasticizers are relatively non-volatile organic substances and will typically be added in manufacturing to the base polymer in the form of a liquid, to modify the flexibility, extensibility and/or processability of the polymer composition, which desired characteristics will be determined by the desired end-use application. Non-limiting general chemical families of common plasticizers useful for polymer modification include: phthalate esters, most commonly, DEHP, (low molecular weight ortho-phthalate) and is the most widely used PVC plasticizer, and DINP, DIDP (high molecular weight ortho-phthalates); aliphatic dibasic acid esters, including glutarates, adipates, azelates and sebecates; benzoate esters; trimellitate esters; polyesters; citrates; bio-based plasticizers, such as epoxidized soybean oil (ESBO), epoxidized linseed oil (ELO), castor oil, palm oil, other vegetable oils, starches and sugars; phosphates; chlorinated paraffins; alkyl sulfonic acid esters and others.

Plasticizers such as dimers may be used herein to enhance the compatibility between the base polymer and the channeling agent. This enhanced compatibility is facilitated by a lowered viscosity of the blend, which may promote a more thorough blending of the base polymer and channeling agent, which under normal conditions can resist combination into a uniform solution. For example, upon solidification of the entrained polymer having a dimer agent added thereto, the interconnecting channels which are formed throughout the base polymer have a greater dispersion and a smaller porosity, thereby establishing a greater density of interconnecting channels throughout the polymer composition.

Optionally, the polymer composition is formed into a granule, a pellet, a film, a sheet, a disk, a seal or cover (of any configuration), a container or a package. In one embodiment according to the invention, the entrained polymer is formed into a film. The size and thickness of the film can vary. Optionally, such film has a thickness of from 0.1 mm to 1.0 mm, preferably from 0.2 mm to 0.6 mm, optionally about 0.3 mm.

### Chlorine Dioxide Gas as Antimicrobial Agent

Chlorine dioxide gas (ClO₂) is a yellow to reddish-yellow gas at room temperature that is stable in the dark but is unstable in light. It is a strong oxidizing agent that under oxidant demand conditions is readily reduced to chlorite (ClO₂—), which is another strong oxidizing agent, and to a lesser extent, chlorate (ClO₃⁻). Chlorine dioxide is a very reactive oxidant, making it capable of inactivating bacterial and viral microorganisms in water and air. Chlorine dioxide gas is known for its ability to affect microorganisms. The primary chemical reaction between the chlorine dioxide and the microorganism is the exchange of an electron between the ClO₂ molecule and the target, initially reducing ClO₂ to ClO₂⁻ (chlorite ion), further electron exchanges will reduce the chlorite ion (ClO₂⁻) to a chlorate ion (ClO₃⁻) and then to a chloride ion (Cl⁻). In aqueous solutions at pH>10, chlorine dioxide will hydrolyze to form chlorate and chlorite ions. In neutral or near neutral solutions (4<pH<10) chlorine dioxide is relatively more stable and exists as a free radical in water. Chlorine dioxide is highly miscible in water up to 60 g/L and is highly unstable in sunlight. Chlorine dioxide has an estimated half-life in water of approximately 25 minutes.

The system and method herein involves disinfecting objects by using embodiments of an engineered polymer composition that incorporates a chlorine dioxide gas forming agent that releases chlorine dioxide in the form of gas as an active antimicrobial agent, in order to inhibit or inactivate microbes or pathogens which may be infectious and/or harmful on the object. Chlorine dioxide is considered to be a broad spectrum antimicrobial agent in that its antimicrobial effect is not targeted to any specific microbe or pathogen, and has been found to be effective against a variety of pathogens. Common viruses include, by non-limiting example, the common cold, influenza, parainfluenza, hepatitis, SARS, (including coronavirus such as Covid-19), measles, rotavirus, virus, respiratory syncytial virus (RSV), Rhinovirus, Ebola, and many others. Common pathogenic bacteria include, by non-limiting example, Salmonella, Escherichia coli, Neisseria, Brucella, Mycobacterium, Mycoplasma, Nocardia, Listeria, Francisella, Legionella, and Yersinia pestis Salmonella, Geotrichum, Campylobacter, Staphylococcus, Streptococcus, Methicillin-resistant Staphylococcus aureus (MRSA), Shigella and many others. Common pathogenic fungi include Aspergillus fumigatus, Aspergillus flavus, Candida albicans, Cryptococcus neoformans, Histoplasma capsulatum, Pneumocystis jirovecii, Stachybotrys chartarum and many others. Copious other antimicrobial agents are known and may be used in combination with chlorine dioxide gas in optional methods and systems described herein.

The system herein combines the use of an antimicrobial polymer comprising a chlorine dioxide gas forming agent that is present within the polymer composition such that upon contact with moisture, it releases chlorine dioxide gas in an amount sufficient to provide antimicrobial effect when the composition is placed into a sealed container or chamber. Prior to such contact with moisture, the chlorine dioxide gas forming agent does not form chlorine dioxide. The released chlorine dioxide gas then functions to decontaminate, disinfect, sanitize, and/or sterilize a desired object. The application of the present disinfection system is not limited to any particular species of microbial agent or pathogen, and it is contemplated that the disinfection system and methods herein will be applicable to the inhibition of any type of microbial infectious agent that is capable of being inhibited by contact with chlorine dioxide gas, as well as to pests, as will be more fully disclosed below.

In optional embodiments, the concentration of the chlorine dioxide forming agent in the entrained polymer composition which will be determined by the desired concentration and strength of the antimicrobial effect (such as the directed substrate and pathogen. The ClO₂ forming agent will form a concentration of the ClO₂ gas in a sealed container. Optionally, the concentration formed can be from 0.001 ppm to 1000 ppm or greater; optionally 0.01 ppm to 3 ppm, optionally 3 ppm to 1000 ppm, optionally 5 ppm to 100 ppm, optionally 10 ppm to 1000 ppm, optionally 30 ppm to 1000 ppm, optionally 60 ppm to 1000 ppm, optionally 100 ppm to 1000 ppm, optionally 10 ppm to 800 ppm, optionally 30 ppm to 600 ppm, optionally 60 ppm to 600 ppm, optionally 100 ppm to 500 ppm, optionally 60 ppm to 200 ppm, optionally 60 ppm to 150 ppm. Optionally, the peak concentration of ClO₂ gas in the container is reached after contact with moisture in a period of 5 minutes to 24 hours, optionally from 5 minutes to 12 hours, optionally from 30 minutes to 10 hours; optionally from 10 minutes to 6 hours, optionally from 10 minutes to 4 hours, optionally from 10 minutes to 2 hours, optionally from one hour to 12 hours, optionally from one hour to 6 hours, optionally from one hour to 3 hours, optionally from one hour to 2 hours.

Optionally, in any embodiment, the amount of chlorine dioxide gas formed by the system herein is present in an amount sufficient to effectuate at least a 1 log base 10 reduction in CFU/g, optionally at least a 2 log base 10 reduction in CFU/g, optionally at least a 3 log base 10 reduction in CFU/g, optionally at least a 4 log base 10 reduction in CFU/g, optionally at least a 5 log base 10 reduction in CFU/g, optionally at least a 6 log base 10 reduction in CFU/g, optionally at least a 7 log base 10 reduction in CFU/g, optionally at least a 8 log base 10 reduction in CFU/g, of at least one type of pathogen.

The polymer composition may be further incorporated or compounded into other materials such as other plastics, paper, glass, wood, metals, ceramics, synthetic resins or combinations thereof.

### Chlorine Dioxide Gas Forming Agent

In the systems and methods herein, a chlorine dioxide gas forming agent is incorporated into engineered polymer compositions. The chlorine dioxide gas forming agent is incorporated into and retained by the base polymer. In alternate embodiments, the chloride dioxide gas forming agents useful herein include chlorite salts, including alkali metal chlorites, alkaline earth metal chlorite or a transition metal chlorite. Non-limiting examples include sodium chlorite, potassium chlorite, barium chlorite, calcium chlorite, magnesium chlorite, or combinations thereof. In optional embodiments, the chloride dioxide gas forming agent comprises chloride salts. In an alternate embodiment, chlorate is also operable herein as a chlorine dioxide gas forming agent, however, it has been determined herein that chlorate does not function as effectively to form chlorine dioxide gas and may therefore not be as desirable for the systems and methods herein.

It is generally believed that the higher the concentration of the chlorine dioxide gas forming agent in an entrained polymer composition, the greater the gas generating and releasing capacity of the final composition. However, too high of a concentration of the chlorine dioxide gas forming agent may cause the entrained polymer to be too brittle. This may also cause the molten mixture of chlorine dioxide gas forming agent, base polymer and channeling agent to be more difficult to either thermally form, extrude or injection mold.

Optionally, in any embodiment, the concentration of the chlorine dioxide gas forming agent in the polymer composition is in the range of 1% to 70%, optionally from 5% to 60%, optionally from 20% to 65%, optionally from 35% to 60%, optionally from 10% to 50%, optionally from 10% to 40%, optionally from 10% to 30%, optionally from 10% to 20%, or optionally about 50% by weight of the total weight of the polymer composition. In addition to modifying or designing the concentration of the chlorine dioxide gas forming agent in the entrained polymer by the concentation of the chlorine dioxide gas forming agent added to the polymer composition, the level of desired chlorine dioxide release can also be controlled by modifying the size parameters of the polymer composition provided to the disinfection system, such as by altering the thickness of the extruded film or sheet for use according to the invention.

Preferred chlorine dioxide gas forming agents herein are compounds or formulations comprising volatile antimicrobial agents that release chlorine dioxide in gas form to function as an antimicrobial material. A volatile chlorine dioxide gas forming agent is generally used in a closed system so that the released chlorine dioxide gas can accumulate within the system and preferably does not escape or at least does not substantially escape. Volatile chlorine dioxide gas forming agents include compounds that produce a gas and/or gas phase such as vapor of chlorine dioxide, when they come into contact with moisture.

In alternate embodiments, the chlorine dioxide gas forming agent that functions as the antimicrobial agent is provided in the polymer composition with a carrier material. Useful herein are chlorine dioxide gas forming agents described in International Patent Application No. PCT/US2019/060937 and in U.S. Publication No. 2019/00335746 A1, each of which is incorporated herein by reference in its entirety as if fully set forth herein. Disclosed in PCT/US2019/060937 is a chlorine dioxide gas forming agent that is provided with a carrier material within the polymer composition that comprises silica or silica gel which is preferably acidified. Alternatively, the carrier material comprises polysulfonic acid. Alternatively, the carrier material comprises a phyllosilicate, such as Montmorillonite clay. Optionally, the chlorine dioxide gas forming agent comprises, consists essentially of or consists of a carrier material (e.g., silica gel), an active compound and a moisture trigger. The carrier material preferably comprises an acidified silica gel having a pH of from 1.4 to 3.1 and is 50% to 90% by weight with respect to the total weight of the antimicrobial releasing agent. The active compound preferably comprises a metal chlorite and is from 5% to 30% by weight with respect to the total weight of the antimicrobial releasing agent. The trigger preferably comprises a hygroscopic compound and is from 2% to 20% by weight with respect to the total weight of the antimicrobial releasing agent. In one optional embodiment, the chlorine dioxide gas forming agent comprises, consists essentially of or consists of from 10% to 15% sodium chlorite, from 5% to 15% calcium chloride, and from 70% to 80% silica gel by weight based on the total weight of the chlorine dioxide gas forming agent. In optional embodiments, the polymer composition comprises sodium chlorite, calcium chloride, silica gel, ethyl vinyl acetate and polyethylene glycol. Preferably, the carrier of the chlorine dioxide gas forming agent has a pH of from 1.0 to 3.5, optionally from 1.4 to 3.1. Effective concentrations of chlorine dioxide gas released by the chlorine dioxide gas forming agent needed to effect antimicrobial growth will range from 0.03 ppm to 1000 ppm depending on substrate and pathogen.

Alternative chlorine dioxide gas forming agents are disclosed and prepared as set forth in U.S. Pat. No. 6,676,850 (incorporated by reference in its entirety. Example 6 of the patent describes a formulation that is particularly suitable as a chlorine dioxide gas forming agent, according to an optional aspect of the invention. The product is provided commercially under the brand ASEPTROL® 7.05 by BASF Catalysts LLC. The product is a formulation of sodium chlorite as the chlorine dioxide gas forming agent, a base catalyst and a trigger. The catalyst and trigger preparations are made separately, then combined with one another, then ultimately combined with the sodium chlorite. The base catalyst is optionally made by first preparing a 25-30 wt. % sodium silicate solution (SiO₂:Na₂O proportion of 2.0 to 3.3 by weight). The solution is mixed into an aqueous slurry of 28-44 wt. % Georgia Kaolin Clay (particle size diameter of about 80% less than one micrometer), wherein the sodium silicate solution is 2 wt. % of the slurry. The slurry is oven dried at 100° C. to generate agglomerates or microspheres of about 70 μm in size. 300 g of these microspheres are impregnated with 280 g of 2.16N sulfuric acid solution. That mixture is then dried at 100° C. Next, the dried mixture undergoes a calcine process at 350° C. for 3 hours, followed by an additional calcine process at 300° C. in a sealed glass jar with the seal wrapped with tape. This mixture forms the base catalyst. Next, 84.6 g of the base catalyst are mixed with 10.1 g of the trigger, dry calcium chloride. This base catalyst and trigger mixture is ground with mortar and pestle at ambient room temperature. The mixture is dried for 2 hours at 200° C. The base catalyst and trigger mixture is then cooled to room temperature in a sealed glass jar with tape wrapped around the seal. Finally, the base catalyst and trigger mixture is combined with 5.3 g of sodium chlorite (the chlorine dioxide gas forming agent). The full mixture is then ground with mortar and pestle at ambient room temperature, thus forming an optional embodiment of a chlorine dioxide gas forming agent. The chlorine dioxide gas forming agent is then deposited in a sealed glass jar with tape wrapped around the seal to preserve it and keep it essentially free of moisture, which would prematurely activate it (to release chlorine dioxide gas).

Accordingly, in one embodiment, an entrained polymer may be a three phase formulation including about 50% by weight of ASEPTROL® 7.05 chlorine dioxide gas forming agent (from Engelhard Corp., Iselin, N.J., USA) in the form of the powdered mixture or another chlorine dioxide gas forming agent, about 38% by weight ethyl vinyl acetate (EVA) as a base polymer and about 12% by weight polyethylene glycol (PEG) as a channeling agent. Alternatively, an entrained polymer may be a three phase formulation including about 50% by weight of a chlorine dioxide gas forming agent, about 43% by weight EVA as a base polymer and about 7% by weight PEG as a channeling agent. Optionally, the powdered mixture further comprises sulfuric acid clay and at least one humidity trigger, optionally calcium chloride.

The chlorine dioxide gas forming agent is provided in an amount sufficient to produce and release the chlorine dioxide gas in a desired concentration sufficient to inhibit microbial growth, such as bacterial, viral, or fungal or other microorgamisms over a predetermined amount of time. According to one aspect of the invention, the amount of chlorine dioxide released can be modified, controlled or designed by altering the concentration of the chlorine dioxide gas forming agent added to the the entrained polymer during manufacture.

In addition to chlorine dioxide gas forming agent as the antimicrobial agent, other known antimicrobial agents, such as alternative antiviral, antibacterial, antifungal agents may be used herein in combination with the chlorine dioxide gas forming agents herein. Such additional antimicrobial agents may include volatile antimicrobial agents, non-volatile antimicrobial agents and combinations thereof. Examples of non-volatile antimicrobial agents include, but are not limited to, ascorbic acid, a sorbate salt, sorbic acid, citric acid, a citrate salt, lactic acid, a lactate salt, benzoic acid, a benzoate salt, a bicarbonate salt, a chelating compound, an alum salt, nisin, ε-polylysine 10%, methyl and/or propyl parabens, or any combination of the foregoing compounds. The salts include the sodium, potassium, calcium, or magnesium salts of any of the compounds listed above. Specific examples include calcium sorbate, calcium ascorbate, potassium bisulfite, potassium metabisulfite, potassium sorbate, or sodium sorbate.

### Controlled Release of ClO₂ Gas

Optionally, the polymer compositions used herein provide a controlled release profile of the chlorine dioxide gas. The amount or rate of chlorine dioxide gas released by the polymer composition herein is modified, in one aspect, by altering the concentration of the chlorine dioxide gas forming agent in the entrained polymer. The amount of chlorine dioxide released can also be controlled by modifying the thickness of an extruded film or sheet herein or the size parameters of the polymer composition in alternate forms in order to control the exact amount of chlorine dioxide gas to be exhuded by and released by the polymer compostion. The amount of the chlorine dioxide gas released will also be controlled by the size of the entrained polymer composition in its final form. For example, in a non-limiting embodiment, the polymer composition of the invention is extruded into film and the film may be further processed into desired size film strips for use (e.g. 10 mm×10 mm strips.).

Optionally, a desired controlled release profile can be achieved by applying a coating to the polymer composition wherein the coating is configured to release the released antimicrobial agent within a desired time frame. The chlorine dioxide gas forming agents may have different coatings applied thereon to achieve different release effects. The coating may be simply layered onto (e.g. physically coated onto), or further adsorbed by, absorbed by or chemically bonded to the polymer composition. Based on predetermined relative release rates of particular formulations (based on loading level of the component materials), the polymer composition may be coated with extended release coatings of varying thicknesses and/or properties to achieve the desired release profile. For example, some polymer compositions will only be lightly coated to initiate almost immediate but slightly delayed formation and release of the chlorine dioxide, such as within seconds, minutes or hours of the exposure to moisture; other embodiments will be more heavily coated or coated with substances that require greater degradation, such that the chlorine dioxide gas forming agent will not be activated until it has been in contact with moisture for days or even weeks.

Another common means utilized to achieve desired control release is spray coating technology, which is known in the art, especially in pharmaceutical indications. For example, pharmaceutical tablets, beads and capsules are sometimes spray coated to control the release rate of active ingredient in order to create extended or sustained release drugs. Such technology may likewise be adapted to apply coatings to the polymer compositions as well as to the chlorine dioxide gas forming agent itself, or to both, in optional aspects of the system of the invention to achieve a desired controlled rate of release of antimicrobial chlorine dioxide gas.

Alternatively, or in addition, a controlled release profile may be achieved by providing a cover layer of a material configured to control moisture uptake into the entrained polymer (which in turn triggers reaction of the released antimicrobial material). For example, the film may include a polymer liner, made e.g., from low density polyethylene (LDPE) disposed on either side or both sides thereof. The thickness of the film and liner(s) can vary. In certain embodiments, the film is approximately 0.3 mm thick and the LDPE liners on either side are each approximately 0.02 mm to 0.04 mm thick. The LDPE liners may be coextruded with the film or laminated thereon.

Alternatively, or in addition, a controlled release and/or desired release profile may be achieved by modifying the formulation of the trigger of the chlorine dioxide gas forming agent. For example, the trigger, when contacted by moisture, liquefies and then reacts with the active component (e.g., sodium chlorite) to cause release of the antimicrobial gas. The trigger may be formulated to liquefy upon contact with moisture at different rates. The faster the trigger liquefies, the faster the release of antimicrobial gas and vice versa. In this way, modification of the trigger is yet another vehicle to provide a desired release rate of antimicrobial gas.

Alternatively or in addition, a controlled release and/or desired release profile may be achieved by altering the pH of a carrier (e.g., silica gel carrier) of the chlorine dioxide gas forming agent. The lower the pH, the more potent the agent. Further, the type and relative amount of base polymer and channeling agent has an impact on the rate at which chlorine dioxide gas is generated and released.

Any combination of the aforementioned mechanisms may be utilized to achieve desired release rates and release profiles of the chlorine dioxide within the headspace of a sealed container or chamber in the system of the invention.

### Activation by Moisture

The chlorine dioxide gas forming agent in the polymer composition herein is triggered (e.g., by chemical reaction or physical change) by contact with moisture. Contact of the chlorine dioxide gas forming agent with moisture will initiate and cause the agent to form chlorine dioxide gas. The activation of the released antimicrobial gas is not initiated until the chlorine dioxide gas forming agent is exposed to the moisture and thereby forms and releases the chlorine dioxide gas.

In a preferred method and system, the design criteria maintains a relative humidity inside the container at less than 5% throughout the shelf life of a piece of personal protective equipment in a state of disinfection based on 30° C./80% relative humidity conditions. Based on the design model, this packaging can support a shelf life of 2.33 years at 30° C./80% relative humidity.

The moisture needed to activate the chlorine dioxide gas forming agent may be supplied by an external source, such as introducing a liquid, vapor, steam or gas that is capable of reacting with the chlorine dioxide gas forming agent in order to form chlorine dioxide. In optional embodiments, the moisture is supplied to the polymer composition by an actuated mechanical or physical means where the moisture comes into direct contact with the polymer composition. Such contact may be achieved in any manner, such as in dipping the polymer composition (for example, in the form of a film strip into water); spritzing or spraying the polymer composition with the moisture agent (for example, from a spray bottle); pouring the moisture agent onto the polymer composition, exposing the polymer composition to water vapor, (for example, from a boiling tea kettle); exposure to humid ambient air (for example, rain or ambient humidity) or by contacting the polymer with a solid surface that comprises moisture thereon, optionally wherein the solid surface is a surface of a wet roller, or by any other contact with moisture.

For example, at such time that a healthcare professional wishes to disinfect a PPE mask, a film strip according to the invention is dipped into or spritzed with water in order to form and release the chlorine dioxide from the entrained polymer composition in order to initiate its antimicrobial functionality, and is then placed into an sealable container with the PPE mask to be disinfected. In alternate embodiments, moisture may be introduced into the interior of the container (before or after being sealed) by piping the moisture into the interior or from releasing the moisture from an adjacent chamber. In an alternate embodiment of a closed chamber or container, the chlorine dioxide gas forming agent is activated by a moisture-releasing object placed into the sealed chamber or container, wherein upon placement into the chamber or container, the object generates moisture that interacts with the chlorine dioxide gas forming agent entrained in the polymer composition, and releases chlorine dioxide into the headspace of the chamber or container. One example of such embodiment is a food packaging container that is sealed in a moisture tight manner to trap moisture within the container generated by a moisture-exuding comestible such as fish (which typically exude moisture during storage at refrigerated temperatures.) According to another embodiment, the polymer composition does not physically contact the object within the sealed container.

Optionally, the entrained polymer composition may further comprise a moisture trigger agent to further accelerate the initiation or rate of reaction and formation of the chlorine dioxide gas by the chlorine dioxide gas forming agent. Optionally, the moisture trigger is a hygroscopic compound. Non-limiting examples of such moisture triggers include, but are not limited to, sodium chloride, calcium chloride, magnesium chloride, lithium chloride, magnesium nitrate, copper sulfate, aluminum sulfate, magnesium sulfate, calcium carbonate, phosphorus pentoxide, lithium bromide and combinations thereof.

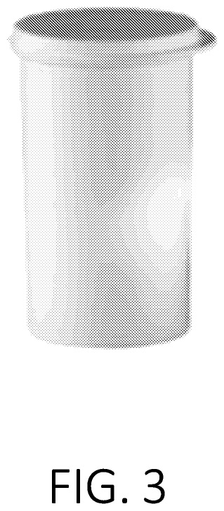

In order to prevent premature initiation of formation and release of the chlorine dioxide gas, it is desired that the polymer compositions optionally be stored in a sealable moisture tight storage vessel prior to their use. Optionally, a desiccant may further be stored along with the polymer composition within the storage vessel to increase sorption of any ambient moisture within the container. The storage vessel can be of any form, shape, material or size. In an optional embodiment, the storage vessel is a plastic vial as shown in FIG. 3, optionally an opaque flip top vial comprising a cap joined to a vial body by a hinge. Preferably, the vessel is moisture tight to protect the polymer compositions from premature formation of ClO₂ gas. Other storage vessels may be plastic bags, cartons, boxes, or any other container comprising a material that is preferably completely or substantially impervious to moisture and/or is moisture tight when closed or sealed.

Optionally, the storage vessel is also entirely or substantially impervious to light in order to prevent undesired photoinitiation of the polymer composition to unwittingly form chlorine dioxide gas prior to the system's intended use. Thereby, the storage vessel is optionally made of a solid material that is impervious to light, or a dark and/or opaque material that minimized contact by the polymer composition with light.

According to an alternate embodiment, a removable liner or covering is placed onto the surface of the polymer composition to prevent exposure and contact with moisture that may be present in ambient air which would cause premature release of the chlorine dioxide active agent. The liner or covering is removed when desired and polymer composition is ready for use, such as by peeling off the covering or liner in order to activate the chlorine dioxide gas forming agent and release the chlorine dioxide.

### Sealing of the Container

It is an important aspect of the method herein that the container, chamber or room into which the object herein to be decontaminated is placed, be closed or sealed. This allows for the chlorine dioxide gas formed by the polymer composition to accumulate within the container, chamber or room and act upon the infectious agent. By sealing the container, chamber or room herein, it is desired that the amount of chlorine dioxide gas that is released by the chlorine dioxide gas forming agent within the container, chamber or room is greater than the exit transmission rate of the chlorine dioxide gas from or through the container, chamber or room.

The sealable container used herein may be sealed by any closing device, sealing means or mechanism, including, but not limited to a seal, a sealable header or resealable header (e.g. on a one gallon sealable plastic bag), a cover, a cap, a lid, a stopper, a door, a plug, a gasket, a washer, a liner, a twist-tie, a bread tab, a bread tag, a clip (such as chip clip), an elastic, an O-ring, a fastener, a combination of the foregoing, or by any other device.

With respect to large objects to be decontaminated, such as medical gowns or medical apparatus, the item may be, for example, placed into a plastic bin having a removable lid or cover. In an alternate embodiment, the polymer composition is provided to the container on the closing device or the closing device itself comprises the polymer composition. Wherein the system or method herein are used to disinfect objects within a room, such as hospital gowns, PPE or medical equipment, the objects optionally are hung and spaced apart from one another to allow the chlorine dioxide gas to more fully engulf or contact the surface of each item.

In alternate embodiments, objects may be placed into a designated disinfection box that is set aside for use according to the invention for the purposes hereof. For example, a medical clinic may designate a standalone cabinet, receptacle, or locker for disinfection purposes. A grocery store may designate a cupboard for use for disinfection of its meat slicing apparatus or a pullout drawer for sanitizing utensils in the prepared foods department. Alternatively, a small chamber or room (having a door) can be used, such as in a hospital setting. The size of a storage compartment, container, chamber or room is not limited by the invention itself herein, but is limited only by practical considerations for desired use, such as the size of the objects desired to be disinfected and the space available in a particular environment such as, for example, a hospital or medical clinic (for disinfection of medical equipment), an elementary school or kindergarten (for disinfection of toys e.g., within a toy box and school supplies), and others.

### Disinfection of Objects

According to one aspect of the invention, the method involves placing an object into a closable or sealable container or into a closable or sealable chamber or into a closable or sealable room together with the polymer composition herein, contacing the polymer composition with moisture to initiate reaction and release of chlorine dioxide gas by the chlorine dioxide gas forming agent, and closing or sealing the container or chamber or room. These steps allow the composition to achieve a formation and release of a desired concentration of the chlorine dioxide gas in the sealed container, chamber or room, so as to effectuate disinfection of the object or room. The ambient air or enviroment surrounding the container, chamber or room preferably remains completely or substantially free of the chlorine dioxide gas during the disinfection process or at a safe level (as considered by GRAS) for human exposure. Unless otherwise specifically stated in this specification or claims in a given instance, the term “safe” means that the amount of chlorine dioxide gas in the ambient environment is “generally recognized as safe” (GRAS) as defined in Sections 201(s) and 409 of the United States Federal Food, Drug, and Cosmetic Act.

With respect to disinfection of objects, after the disinfection is completed, when the object is removed from the container or chamber, the chlorine dioxide gas dissipates, breaks down or chemically converts into the environment around the object and becomes completely or substantially undetectable with instrumentation immediately or within a short period of time, (e.g. a few minutes; optionally 1 minute; optionally within 5 minutes) after removal from the sealed container or chamber. The physical integrity of the object remains essentially unaffected and the object becomes readily available and safe for re-use. Optionally, this process may be repeated multiple times without adversely affecting the physical integrity or performance of the object.

The amount of time needed to complete disinfection herein is not limited. In preferred embodiments, the object to be disinfected will be maintained in the closed container for a period of 10 minutes to 12 hours, optionally from 10 minutes to 6 hours, optionally from 10 minutes to 4 hours, optionally from 10 minutes to 3 hours, optionally from 10 minutes to 2 hours, optionally from 10 min to 1 hour; optionally from 1 hour to 4 hours, optionally from 1 hour to 2 hours; optionally from 2 hours to 3 hours. As disclosed herein, the bioburden reduction cycle for N95 PPE masks is preferably approximately 10 hours in a one gallon sealed plastic bag.

The system and method of disinfection herein can also be used in conjunction with (e.g., either before or after) or as a replacement for any of a variety of sterilization methods, such as, but not limited to, heat sterilization (i.e., wet/steam or dry), filtration sterilization, radiation sterilization, pressure sterilization, and/or chemical sterilization. Optionally, one or more methods can be combined in any order, whether in series or in parallel or with other sterilization methods.

### N95 Respirator Masks

A preferred use of the system and method herein is the disinfection of N95 or N95-equivalent personal protective equipment (PPE) face masks. A method of disinfecting an N95 respirator mask as defined by the National Institute for Occupational Safety and Health of the United States (NIOSH). Typically, but not always and not necessarily, such masks comprise polypropylene fiber.

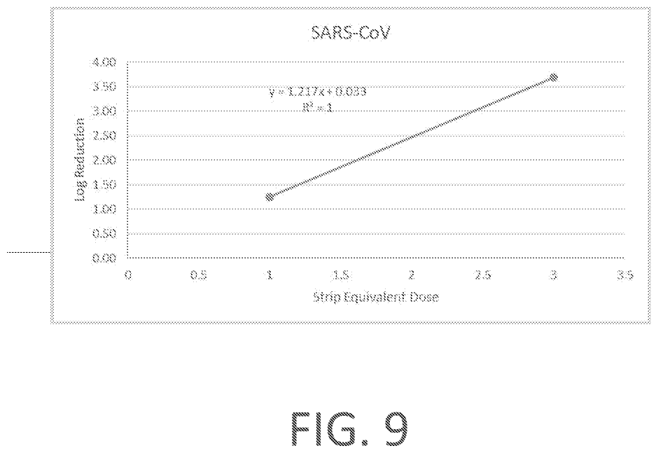

The method comprises the steps of: (a) placing at least one N95 respirator mask into a sealable container, optionally a polypropylene or polyethylene plastic resealable zipper storage bag that is optionally a quart to two gallons in volume or a plastic snap-top food storage container that is optionally a quart to five gallons in volume, a headspace being formed of a portion of the interior space of the container that is not occupied by the mask, e.g., as shown in FIG. 9; (b) placing into the container a polymer composition comprising: (i) a base polymer; (ii) a chlorine dioxide gas forming agent; and (iii) a channeling agent that forms channels though the base polymer; (c) contacting the polymer composition with moisture to form chlorine dioxide gas; and (d) closing the container completely or sufficiently enough to allow the chlorine dioxide gas to accumulate in the headspace, wherein the chlorine dioxide gas disinfects the mask. Optionally, this system provides a chemical indicator that allows the user to physically mark and visually confirm that the entrained polymer strip has been activated or has not been activated. Optionally, the chemical indicator comes pre-marked on the entrained polymer strip.

Optionally, only a single mask is provided in the container at a time, however, two or more masks can optionally be provided. Optionally, the plastic bag is hung during the disinfection process in order to optimize distribution of the chlorine dioxide gas in the headspace of the bag. In an optional embodiment, the chlorine dioxide gas accumulated in the closed container for face mask disinfection is present in a concentration of from 1 ppm to 30 ppm; optionally from 4 to 18 ppm over a period of 1 minute to 40 minutes; and optionally it reaches a peak concentration of at least 15 ppm to 25 ppm in 20 minutes and decreases to less than 3 ppm by 60 min. Alternatively, the chlorine dioxide gas accumulated in the closed container for mask (or other PPE) disinfection reaches a peak concentration of at least 30 ppm, optionally at least 60 ppm, optionally at least 80 ppm, optionally at least 120 ppm, optionally at least 180 ppm, optionally 60 ppm to 1000 ppm, optionally 60 ppm to 500 ppm, optionally 60 ppm to 250 ppm, optionally 60 ppm to 180 ppm, optionally 80 ppm to 150 ppm. Preferably, the peak concentration is reached sometime from 5 minutes to three hours after initiation of the process though a complete disinfection cycle can take up to 10 hours in abundance of caution. The chlorine dioxide gas permeates through the mask, especially when only a single mask is provided in the container at a time.

Optionally, a entrained polymer strip 10 mm×10 mm is provided. It was determined that a single 10 mm×10 mm strip of polymer composition used according the method of the invention can achieve a greater than 99.999% reduction in Feline calicivirus, ATCC VR-782 on an N95 PPE respirator mask.

In a particular embodiment, provided is a system pursuant to U.S. FDA testing protocol PEUA200320. An entrained ClO₂ forming polymer is provided with a qualitative chemical indicator to indicate the effect of the ClO₂ gas in a closed sealed one gallon bag as an internal process monitor, as will be more fully disclosed in the Examples.

The system and method herein have been determined to be effective with any N95 respirators constructed from polypropylene and/or polyester, including, for example, the following brands and models (from the 3M Company of Minnesota, U.S.): 3M™ Particulate Respirator 8511, (made of polypropylene filter and polyester shell; 3M™ Particulate Respirator 9211 (made of polypropylene filter and polypropylene cover web), and 3M™ Particulate Respirator 1860, (made of polypropylene filter and polyester shell).

The decontamination system herein presents a multitude of benefits. It allows for medical professionals to retain the same respirator for re-use eliminating the potential for cross-contamination between users. It can be executed at the point of care without shipping the respirators to an external site for processing. Because the respirators do not need to leave the point of care and are returned to the same user, there is no need for repackaging or relabeling of the unit. The system provides access to serve rural remote health care facilities that currently do not have access to bioburden reduction or decontamination systems. According to an embodiment, the total bioburden reduction time is 10 hours. It is possible for respirators to undergo bioburden reduction and be made available to the user in the time between the end of a shift of the healthcare professional to the beginning of a new shift, thus reducing the number of respirators that need to be assigned to a single medical professional on a rotating basis.

Unlike traditional sterilization processes, this procedure scales with demand without the need for revalidation. This technique is procedurally and economically viable in small and large institutions alike. The process does not change if a facility needs to process bioburden reduction on one respirator or on multitudes. This method does not require the construction or acquisition of specialized training or equipment for this process. It can be executed in any room in a point of care facility without supporting utilities or facilities.

### Degradation of Product

An important aspect of the invention is that the system as used herein does not cause any significant damage or performance or use degradation to the object being decontaminated. With respect specifically to N95 PPE face masks, the disinfection, decontamination, sanitation or sterilization of the N95 masks does not meaningfully degrade filtration performance or degrade any mask component. The disinfection system can be used multiple times on the same piece of PPE without any significantly measurable degradation in filtration performance capacity. Independent testing of the disinfection system herein was performed on N95 respirator masks at Auburn University (Alabama, USA) to validate the integrity of the masks upon treatment with the system. After ten (10) repeat cycles of the methodology and system, it was measured that the filtration efficiency of the masks was retained at over 95%. It was also found that the 95% integrity in filtration was equal to that of the control samples which did not undergo any disinfection treatment. Further testing by the Georgia Institute of Technology (Atlanta, Ga., USA) and SGS Laboratories (SGS S.A., Geneva, Switzerland), pursuant to protocol of the U.S. National Institute for Occupational Safety and Health (NIOSH) confirmed that after twelve (12) treatments with the disinfection method and system herein, N95 masks showed no ascertainable degradation. In addition, the elastomer straps or bands disposed on the mask and the amount of stretch performance of the elastomer straps or bands was also unchanged or not substantially changed after up to 10 cycles of disinfection with consistent bioburden reduction results. Thereby, it is contemplated that the disinfection system herein can be used on an N95 respirator mask or N95-equivalent type mask at least ten (10) times before the mask could be discarded due to safety concerns out of an abundance of caution. As such, an addition benefit is that the system potentially reduces respirator consumption by an order of magnitude.

In a similar aspect, it is contemplated that the disinfection system herein does not cause significant degradation of other objects after several cycles of use (e.g., at least 5 or at least 10 cycles), including other medical tools or other products.

### Environment Surrounding the System

Another important aspect of the invention is its minimal exposure to the environment surrounding the system of the chlorine dioxide gas. After the object is treated by the disinfection system, the chlorine dioxide gas dissipates from the object into the atmosphere or local environment outside of the object or undergoes conversion to a chemically inert substance. In the case of personal protective equipment, such as the N95 or N95-equivalent PPE mask, for example, the object becomes safe for use by the wearer while retaining its effectiveness against infectious agents. The amount of chlorine dioxide gas after disinfection and removal of the PPE from a sealed container is completely or virtually undetectable and the mask becomes safe for use or re-use.

Optionally, the amount of chlorine dioxide gas in the ambient environment around the container is less than 0.01 ppm the entire time that the method is performed and optionally immediately after removal of the object from the container, optionally, within 1 minute after removal. Optionally, the amount of chlorine dioxide gas in the ambient environment around the container is considered Generally Recognized as Safe (GRAS) pursuant to Sections 201(s) and 409 of the United States Federal Food, Drug, and Cosmetic Act the entire time that the method is performed and optionally immediately after removal of the object from the container, optionally, within 1 minute after removal.

During the decontamination process, it is possible that a user might open the container prematurely or the container might be accidentally punctured or experience a hole. Tests were conducted to determine the chlorine dioxide gas concentration level a user may experience while the bioburden reduction cycle attains peak concentration. The OSHA guidelines has set an 8-hour Threshold Limit Value (TLV) of 0.1 ppm for occupational exposures to chlorine dioxide in order to minimize the potential for respiratory tract irritation and bronchitis. The test acceptance criteria was set for results to be below 0.1 ppm as the acceptable level. It was found that at a one foot height directly above the one gallon sealed bag, there is no ClO₂ measurement readings when the bag is open or has a small hole when the peak concentration is attained at 30 minutes. These results demonstrated that there exists an adequate degree of safety for the user during exposure to the system herein during a bioburden reduction process if the container, such as a plastic bag, unseals or a hole is generated in the bag.

### Chlorine Dioxide Exposure Limits

In some circumstances, the disclosed systems and methods may be used to disinfect a confined space configured to accommodate human beings, such as a room or a car. In some embodiments, it may be desired to disinfect the confined space even while living persons are present. In such situations, the chosen concentration of the chlorine dioxide gas forming agent will be an amount that corresponds to the amount of chlorine dioxide gas that is released and is regarded or mandated as safe for human use by guidelines set forth by the U.S. Food and Drug Administration (FDA), the Centers of Disease Control and Prevention (CDC) or by another relevant governmental agency. The CDC sets forth the safe exposure limit for an individual for chlorine dioxide gas to be 0.1 ppm over an average of a 10-hour work shift, and 0.3 ppm for an average of 15 minutes. The Occupational Health and Safety Administration (OSHA) guidelines provide that a person can be exposed to 0.1 ppm of chlorine dioxide gas for up to 8 hours per day up to 5 days per week (40 hour workweek equivalent) (0.28 milligrams per cubic meter [mg/m3]). The American Conference of Governmental Industrial Hygienists (ACGIH), which recommends occupational exposure limits for chemicals, has established an 8 hour Threshold Limit Value (TLV) of 0.1 ppm for occupational exposures to chlorine dioxide in order to minimize the potential for respiratory tract irritation and bronchitis. ACGIH has also recommended a Short-Term Exposure Limit (STEL) of 0.3 ppm (0.83 mg/m3). These benchmarks mirror the standards of the U.S. National Institute for Occupational Safety and Health (NIOSH) and OSHA. The TLV is a level which may be safely inhaled by workers for repeated full shift exposures (8 to 10 hours per day) throughout their work-life without significant adverse health effects. The USEPA's Reference Concentrations in air (RfCs), which are more stringent than the OSHA standards, are 0.003 ppm for workers (long-term repeated workplace exposures), and 0.05 ppm for consumers (for brief periods). Thus, in certain embodiments, the level of released chlorine dioxide is directed to these agency mandated or recommended safety profiles, and such embodiments are formulated well below any levels of safety concern.

Optionally, the loading level or concentration of the chlorine dioxide gas forming agent within the entrained polymer composition used in the disinfection system herein can range from 10% to 80% by weight with respect to the total weight of the entrained polymer. The more highly loaded embodiments may find use for disinfection or sterilization of other personal protective equipment, (PPE) such as, earplugs, (including single use earplugs and molded earplugs), earmuffs, ear defenders, safety spectacles, safety glasses, goggles, laser safety goggles, welding shields, face shields, masks, respiratory protective equipment, helmets, hard hats, hand gloves, aluminized gloves, leather gloves, aramid fiber gloves, synthetic gloves, fabric gloves, coated fabric gloves, butyl gloves, latex rubber gloves, neoprene gloves, nitrile gloves, leggings, shin guards, foot guards, toe guards, safety shoes, hazmat suits and high visibility clothing. The system and method will further provide much needed use in disinfection of medical devices and equipment such as blades, scalpels, syringes, needles, scissors, cannulas, intravenous sets, implants, test kits, inhalers, catheters, probes, endoscopes and myriad of others. Scientific devices and equipment such are used in hospitals, dental offices, and research laboratories may include petri dishes, flasks, biological safety cabinets, and a myriad of other medical and scientific objects wherein the cleansing of such equipment is not necessarily limited by exposure to or contact with a certain level of chlorine dioxide.

### Color Indicator

Optionally, a color indicator is incorporated into the polymer composition that is useful to visually demonstate that chlorine dioxide gas has been formed and optionally that disinfection of the object has been achieved. Such color indicator may be applied during the manufacture of the polymer composition or may be applied later, e.g., at the time of use. For example, a kit comprising one or more strips of the polymer composition may also include a permanent marker that is used to apply an ink mark to the surface of the polymer strip, wherein the ink mark fades, changes color or becomes visually imperceptable after the polymer composition has been triggered with moisture and has released chlorine dioxide gas to a certain extent. Applicants have found that certain inks behave differently than others in this respect.

For example, a Sharpie™ Electric Pink #1927338 permanent marker had been used to apply an ink marking as a color indicator to the surface of samples of the polymer composition. Some samples of the composition were then activated with water (i.e., briefly dipping in liquid water) and left to release chlorine dioxide gas in a chamber while other samples were left alone and not activated. After 15 minutes, the markings on the activated samples had faded significantly and after 45 minutes, the markings were no longer visually perceptible. By contrast, the pink markings on the samples that had not been activated were of the same color and intensity 60 minutes after being so marked. A purple permanent marker of the same brand was also used on samples of the polymer composition that were activated with water. After 15 minutes, the purple ink had faded to a pink color and after 60 minutes, the ink still remained as a pink color of approximately the same intensity as perceived at the 15 minute mark. These tests demonstrated that selection of color and ink may provide different effects, depending on what is desired in a visual indicator for a given application.

Optionally, instead of or in addition to the color indicator within the polymer composition, the method or the disinfection system herein further incorporates a standalone chlorine dioxide gas indicator within the container to measure the concentration of chlorine dioxide gas inside the container or to otherwise indicate that chlorine dioxide gas has been formed and optionally that disinfection of the object has been achieved. Such a standalone indicator may provide visual indicia akin to a pregnancy test or litmus test. According to a particular embodiment, provided is a color indicator in the form of a marker that is capable of writing on a strip of polymer film herein. Any color marker is contemplated and the marking is not necessarily limited to any specific color, although as set forth above, different colors may have different effects.

The color indicator may indicate a change in color or a change in shade of the same color, or of at least a portion of a change in color or shade of the polymer composition, wherein prior to contact with moisture the color or shade is different than the color or shade of the at least a portion of the polymer composition after formation of the chlorine dioxide gas. Optionally, the color or shade of at least a portion of the polymer composition or the demarkation with the marker appears different or faded after formation of the chlorine dioxide gas compared the color or shade prior to contact of the polymer composition with moisture. For example, the color indicator may include a solid and dark blue, green, purple, pink or red marking that fades after a predetermined amount of chlorine dioxide gas has been formed. The color is not limited.

Alternatively, one or more color indicator strips may be provided within the container or chamber to indicate when disinfection or sterilization has been achieved.

### Alternative Uses of Disclosed Systems and Methods

The disinfection system herein will find use in innumerable fields and indications, including in areas of health care, surgery, dentistry, cosmetology, veterinary practice, research laboratories, clean rooms, construction sites, transportation, other vehicles of travel, social gathering facilities, stores and shopping malls, offices, schools, concert halls, and any situations and settings in which persons may have concern relating to airborne transmission of pathogens, as well as disinfection of any object or product, regardless of size, that is capable of being enclosed and disinfected, as long as a container or chamber large enough is available for its use in connection with the system herein. The method of the invention will be operable as long as the container or chamber contains sufficient space around the object or parts or at least significant parts of the object to allow the formation of chlorine dioxide gas and contact with the chlorine dioxide gas by the object in the interior of the container or the chamber.

### Sanitation of Consumer Products

The method of the disinfection system of the invention will find use in cleansing, disinfection, decontamination, sanitation and/or sterilization for a plethora of products, items, objects, and/or spaces. Non-limiting examples of objects that can benefit from the systems and methods described herein include kitchen wares, such eating utensils and implements (e.g., forks, knives, spoons), cooking utensils, oven mitts, serving dishes, drinking glassware, mugs, cutting boards, measuring cups, kitchen tools, kitchen shears, can openers, wine openers, mixing bowls, potato mashers, tongs, colanders, graters, whisks, vegetable peelers, rolling pins, blenders, immersion blenders, slicers, mixers, food scales, pots, sauce pans, frying pans, skillets, roasting pans, Dutch ovens, cooling racks, food storage containers, juicers, tea kettles, food storage containers, and many others.

Another particularly useful embodiment is the disinfection of children's toys and sports equipment. A particularly preferred embodiment is the disinfection of mobile telephones which can be disinfected according to the method of the invention, and accompanying cell phone accessories such as cases, headphones, cables and others. Another particularly desired use will be for computer hardware or components (e.g., keyboard, mouse, other handheld equipment, and semi-conductor chips) which are often difficult to clean due to the more sensitive nature of the electronic components to contact with the typically used cleaning products. Another particularly useful embodiment is the disinfection of eye glasses, sun glasses and contact lenses. Another particularly useful embodiment is in use with personal care products such as toothbrushes, hair brushes, towels and many others.

A tremendous need also exists for the disinfection of cosmetic products and accessories, such as lipsticks, eyeshadows, blushes, powders, cosmetic brushes, cosmetic bags, and myriad of others, as these products come into continuous contact with microbes and are known to proliferate on the product and may cause harm. Disinfection of jewelry and hair accessories is also desired (e.g., watches, rings, earrings, hair clips and many others). Of particular use may also be the disinfection of packages as more and more online shopping is carried out by consumers and packages are handled by manual workers as orders are filled and shipped and are subsequently further handled by delivery persons when being delivered. Another particularly useful embodiment is the disinfection of currency, as currency is extensively handled and is generally known to carry numerous germs. Many other applications of the technology are apparent.

The presently disclosed system can be beneficial and useful for disinfection of articles formed of any of a variety of materials, such as, but not limited to, plastics, polymers, metals, alloys, stone, wood, paper, glass, fiberglass, alloys, ceramics, resins, fabrics, textiles, nylons, elastomers, and any other materials or combinations thereof.

### Autoclaves

In yet an alternate embodiment, the method and system of the invention are used in an autoclave, in combination with an autoclave or as a standalone chlorine dioxide gas autoclave. Generally, an autoclave is a machine used to perform industrial and scientific processes requiring elevated temperature and pressure in relation to ambient temperature and pressure. Autoclaves are used in medical applications to perform sterilization, typically steam sterilization. According to optional embodiments, the polymer compositions herein are loaded for high dose release of chlorine dioxide gas, activated by moisture and placed into a dedicated tightly sealable autoclave with the object to be sterilized. The object within the autoclave undergoes sterilization or disinfection when the door of the autoclave is closed. Thus, as used in the context of optional embodiments of the invention, the autoclave may be a tightly sealable dedicated chamber for sterilization without typical accoutrements of medical autoclaves, such as means for increasing temperature and pressure. In this way, an autoclave according to optional embodiments is less complex and expensive than a typical steam sterilization autoclave, for example. Optional use of autoclave systems according to the invention may be for medical, surgical or dental equipment that are typically sterilized with standard autoclaves. But a further advantage of the autoclave system herein is that it may be used to sterilize items that cannot withstand the heat, steam and/or pressure conditions of a standard autoclave.

In a broad sense, therefore, the autoclave functions as the container or chamber for the retention of the chlorine dioxide gas. The chlorine dioxide gas accumulated within the autoclave can be removed by activating a ventilation system inside the autoclave chamber venting the gas in a safe manner to the outside of the autoclave and/or room or the chlorine dioxide gas is allowed to dissipate from the autoclave chamber over a given time period, as set forth in some examples, below. Optionally, the autoclave will include a chlorine dioxide meter that provides real-time and instantaneous chlorine dioxide gas concentration readout and display. The autoclave is operable to be locked shut to seal the object within the chamber during disinfection, the sealable chamber optionally being from 20 liters to 2000 liters in volume.

The autoclave chamber optionally comprises a vent configured to open to release chlorine dioxide gas from the chamber to a location outside of the chamber and to close to prevent release of chlorine dioxide gas from the chamber. Optionally, the vent is configured to open and close automatically upon reaching a predetermined condition within the chamber, automatically upon reaching a predetermined time parameter or upon manual actuation.

The chlorine dioxide gas sensor within the chamber is configured to detect chlorine dioxide gas concentration within the chamber, the sensor being configured to transmit a signal indicative of the chlorine dioxide gas concentration within the chamber at a given time to a readout display, optionally wherein the chamber comprises the vent which is configured to open and close based on detected concentration of chlorine dioxide within the chamber, optionally wherein the door is configured not to unlock until detected concentration of chlorine dioxide within the chamber has reached a predetermined safe level for human exposure.

The autoclave system herein achieves a high-level disinfection or sterilization including sporicidal efficacy in a short period of time, within seconds, minutes, or hours, destroying the most resistant pathogens and spores, including, but not limited to Bacillus subtilis, Clostridium difficile, coronavirus, COVID-19, influenza, Mycobacterium tuberculosis, Mycobacterium avium, Polyomavirus SV40 (surrogate of HPV), Vancomycin-Resistant Enterococcus faecium (VRE), Klebsiella pneumonia, Escherichia coli (E. coli), Aspergillus brasiliensis (formerly niger), Candida albicans, Adenovirus, Staphylococcus aureus, Human Immunodeficiency Virus (HIV), Hepatitis B virus, and Hepatitis C virus.

### Packaging

In yet an alternate embodiment, the disinfection system herein will find use in maintaining a decontaminated environment in the packaging, distribution and storage of products where a sterilized package environment may be of benefit. Non-limiting examples include the packaging, distribution and storage of electronic components, cosmetics, pharmaceutical products, and others. For example, cosmetics are often placed into a plastic bag before being put into a box. A chlorine dioxide generating polymer composition as described herein may be wetted and then disposed into the bag with the cosmetics for commercial distribution. In such application, the moisture applied to the polymer composition may optionally be provided via a wet roller mechanism during an in-line packaging process. In this way, products may be disinfected in transit.

### Room and Car Decontaminants

An alternate embodiment of the invention is provided herein as a room decontamination system and method. The antimicrobial polymer compositions herein are designed for use to disinfect the interior of a chamber. Optionally, the disclosed technology can be utilized and beneficial for any space that can be at least temporarily confined (i.e. enclosed and/or sealed). Any objects within the room will also come into contact with the chloride dioxide gas and be at least partially or fully disinfected.

In such embodiments, the concentration of the chlorine dioxide gas forming agent will be desired in concentrations greater than used for the disinfection of objects, since a larger amount of chlorine dioxide gas is necessary for larger volume of space such as a room. Since the amount of chlorine dioxide release is not limited, as described hereinabove, use of the system will be dictated by the volume, time and specific load of chlorine dioxide release from the polymer composition available for decontamination in order to decrease the bioburden within a room or other such confined space.

With respect to room decontamination, two particular embodiments are contemplated herein, or a combination of the two as will be apparent. According to one embodiment, room disinfection is achieved while persons (and/or animals) are or may be present in the room. In such embodiments, the amount of chlorine dioxide released should be that as considered safe for human exposure as disclosed herein, yet sufficiently effective to decontaminate the room including air and optionally surfaces and objects therein. Such safety guidelines are discussed heretofore. Given the specific limits on chlorine dioxide gas deemed safe for humans, the controlled release of such gas as provided by polymer compositions of the invention is particularly desirable. Optionally, one or more ventilators may be provided in the room during the disinfection cycle to facilitate distribution and consistent concentration of the chlorine dioxide gas throughout the room.

According to a second embodiment, room disinfection is effectuated at high concentrations of chlorine dioxide gas when no persons (or animals) are present in the room. The polymer compositions will be placed into the room, for example as large sheets or many pieces of film, contacting the sheets or film with moisture, or introducing moisture into the room via an alternate source, and thereby enabling a powerful burst of the chlorine dioxide gas and exposure of the air, surfaces and objects within the room to the chlorine dioxide gas for disinfection, decontamination, sanitation or sterilization of microbes. Optionally, such sterilization may be performed overnight. The chloride dioxide gas in the room accumulates to a concentration of at least 0.03, optionally 0.1 ppm, optionally, 0.2 ppm, optionally 0.3 ppm, optionally 0.5 ppm, optionally 1.0 ppm, optionally 3.0 ppm, optionally 5.0 ppm, optionally 10 ppm, optionally 30 ppm optionally 50 ppm, optionally 100 ppm, optionally 1000 ppm. The room is ready for re-entry and use once the chlorine dioxide gas is removed or falls to a concentration considered safe for human exposure, as discussed hereinabove.

Use may be found, for example, in school classrooms, gymnasiums, hotel rooms, corporate offices, conference rooms and hallways, hospital rooms, bathrooms in homes or public buildings, and many other spaces as is apparent.

In an alternate embodiment, a car decontaminant is provided by placing the polymer composition herein inside a car, activating the composition by water, shutting the car doors and windows for some time, allowing chlorine dioxide to accumulate inside the sealed interior of the car thereby disinfecting any microbes on the surfaces inside the car, and helping to cleanse the air inside the car by inhibiting potentially harmful microbes. Once the doors and/or windows of the car are opened, the chlorine dioxide that had accumulated inside the car dissipates into the atmosphere and the car is safe for use. Such application will be particularly useful for car-hailing such as taxis, Uber®, Lyft® and other car-ride and car-share services. Or this may be implemented to decontaminate the interiors of rental vehicles between uses.

Use will also be found for decontamination and sterilization of the interior of ambulances to help to prevent pathogens from infecting potentially immuno-compromised patients being transported therein, as well as ambulance personnel. Application in buses, trains, boats, airplanes and other vehicles of travel will also minimize potential spread of infectious diseases by using the method of this invention on a regular basis.

### Deodorizers

Chlorine dioxide gas has also been recognized as being highly effective at eliminating odors. In alternate embodiments, the system herein is used as a space freshener, deodorizer, air sanitizer or odor diffuser in accordance with the method herein for the purpose of eliminating odors emanating from an object or in ambient air in a container (for example, a shoe box), a compartment (for example, a dresser drawer), a chamber (for example, a kitchen pantry) or a room (for example, a bathroom). Examples of odors treatable herein include, but are not limited to, odors resulting from smoke, water damage (possibly fungal activity), pets, foods, paints, chemicals, and many others.

The entrained polymers herein can further optionally include one or more additional odor-eliminating or masking agents, odor-neutralizers, odor-emitters, deodorizers, disinfectants, chemical neutralizers, smoke-absorbing agents, and anti-nausea agents. Non-limiting examples of such compounds include zeolites, activated carbon, and alumina that are capable of removing a wide range of different materials and odors and impart air filtering capabilities.

The room decontaminants and room deodorizers to be used according to the method herein can be provided in various shaped forms, not limited to sheets, film or any other structure. For example, the decontaminants and deodorizers for use herein may be molded from the entrained antimicrobial polymer compositions into any desired shape, such as ornamental shapes for the consumer market, into animal shapes for use in children's rooms, into geometric designs for use in office spaces, and any other shapes, forms, figures or designs.

### Air Filters

The decontamination or disinfection system herein can be incorporated into filtration devices used to purify or sterilize air. An air decontamination filter of the invention comprises a chlorine dioxide gas forming polymer composition, the polymer composition having (a) a base polymer, (b) a chlorine dioxide gas forming agent, and (c) a channeling agent forming channels though the base polymer; wherein, contact of the polymer composition with moisture in air passing through the filter is configured to form chlorine dioxide gas at a sufficient concentration to decontaminate the air.

Examples of filtrations devices include, for example, filters that may be used in air filtration systems, air conditioners, heaters, high-efficiency particulate air (HEPA) filters, vacuum cleaners, refrigerator filters, range hood filters, and other such applications. For example, strips of the antimicrobial polymer compositions can be placed onto the filter and once activated by moisture that comes into contact with the polymer composition from the air moving through the filtration device, the chlorine dioxide gas is formed and released into the surrounding environment. Alternatively, the polymer compositions can be provided as filter liners. Alternatively, the polymer compositions can be manufactured directly into reusable and disposable filters.

### Pest Control

In addition to antimicrobial properties, as discussed herein, chlorine dioxide gas is noxious to human and animal life at high concentrations. In yet a further embodiment, the chlorine dioxide gas forming system herein is adapted for use in pest control management, specifically for use with rodent and insect traps. Particular uses will be for the immobilization and/or extermination of pests such as insects, including but not limited to moths, mosquitoes, flies, ants, cockroaches, bed bugs, termites, crickets, locusts, wasps, aphids, woodworms, beetles and caterpillars. Other pests may include rodents such as mice, rats, squirrels, chipmunks, rabbits, raccoons, possum, skunks, and snakes.

The apparatus and method for trapping and exterminating a pest comprises: (a) a chamber having at least one inlet to provide entry into the chamber by the pest, optionally wherein the inlet is sealable; (b) a chlorine dioxide gas forming polymer composition provided within the chamber, the polymer composition comprising: (i) a base polymer, (ii) a chlorine dioxide gas forming agent, and (iii) a channeling agent forming channels though the base polymer; wherein, after entry of the pest into the chamber, the pest becomes trapped in the chamber and chlorine dioxide gas is formed and accumulates inside the chamber in a concentration sufficient to exterminate the pest.

Moisture needed to activate chlorine dioxide gas release is introduced into the system by the pest's breathing. Alternatively, a capsule containing a liquid is retained in the chamber that is dislodged once the pest enters the apparatus. Other means of providing moisture are operable.

The invention will be illustrated in more detail with reference to the following Examples, but it should be understood that the invention is not deemed to be limited thereto.

### EXAMPLES

Antimicrobial entrained polymer film was prepared and used according to various aspects of the disclosed concept to test microbial inhibiting activity and other features. The film was prepared by extrusion as a three phase entrained polymer film including a chlorine dioxide gas forming agent in the form of a powdered mixture. The powdered mixture included a silica gel carrier having a pH of about 1.6, which was present at 77% of the total weight of the powdered mixture. The powdered mixture further included calcium chloride, present at 10% of the total weight of the powdered mixture and sodium chlorite, present at 13% of the total weight of the powdered mixture. The polymer composition comprised 50% by weight of the chlorine dioxide gas forming agent, 43% by weight of ethylene vinyl acetate as base polymer and 7% by weight of polyethylene glycol (PEG) as channeling agent. The formulation was provided as a film having a thickness of about 0.3 mm and cut into strips measuring as described in the examples below. The film formulation as described herein is considered one exemplary non-limiting embodiment of an entrained polymer for use with the disclosed disinfection system and method. The film strips remain dry until ready for use. The film was then completely submerged by tweezers into water for one to two seconds. As described above, the film was triggered by moisture of the water to release chlorine dioxide (ClO₂) gas. The film strips were then tested as set forth in each example below, at room temperature between 20° C. and 25° C. (68° F. and 77° F., not to exceed 86° F.).

### Example 1—Efficacy of ClO₂ Gas in Sealed Bag

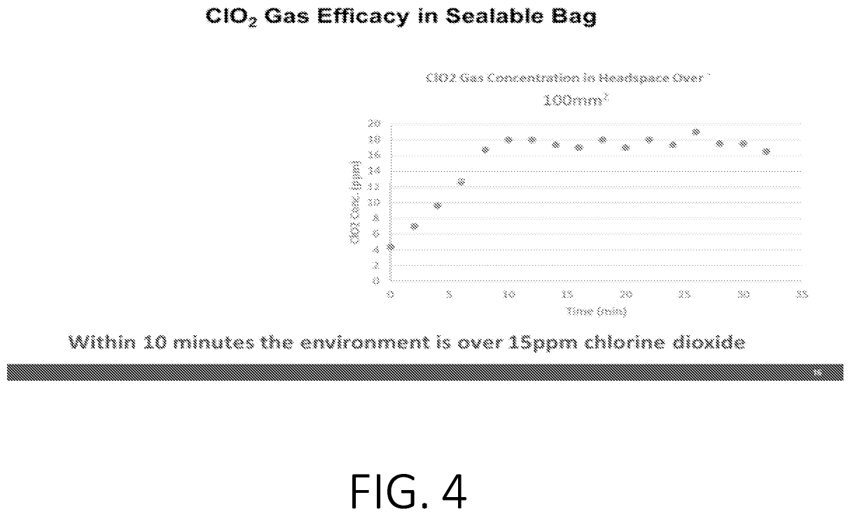

Strips of film according to the formulation above were prepared and cut into 10 mm×10 mm strips. The strips were fully submerged by dipping into water and the wet film was placed immediately into a one gallon bag having a resealable zipper seal and the bag was sealed. The gas efficiency or release profile of the ClO₂ gas was measured with a ClorDisys EMS™ system of ClorDisys Solutions Inc., (Branchburg, N.J., U.S.A.) The EMS™ system uses split-beam reference compensation at a second wavelength to provide precise read and display of the concentration measurement. The experiment was repeated at least three times. An average of the trials was calculated and set forth in FIG. 4. The figure shows a quick release and accumulation of approximately 15 to 20 ppm of ClO₂ gas in the sealed bag within approximately 10 minutes from T₀ (time zero being the time that the bag was sealed.) The concentration of ClO₂ gas remained approximately at this level within the sealed bag for at least 35 minutes.

### Example 2—Comparison of ClO₂ Gas Concentration in Different Bags

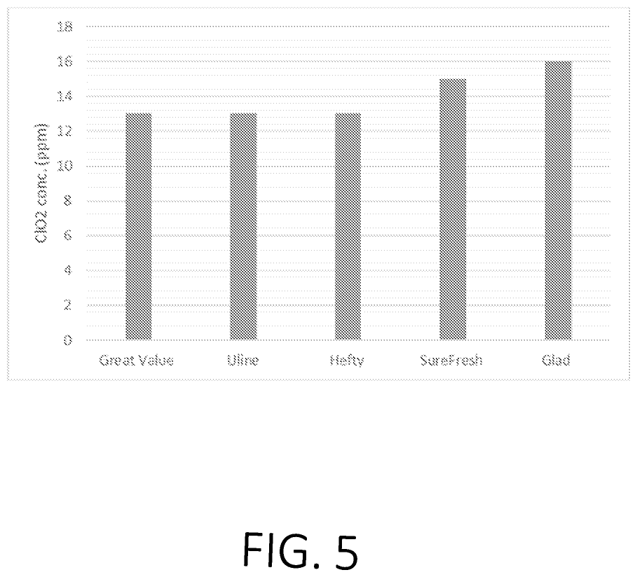

The concentrations of ClO₂ gas in different types of (re)sealable plastic bags were tested for comparison. Strips of entrained antimicrobial polymer film were prepared and tested in accordance with the method as set forth in Example 1. The bags were sealed and the concentration of ClO₂ gas in each bag measured. Table 1 and FIG. 5 provide the peak dose concentration of the ClO₂ gas inside each of the sealed containers. The results indicate a relatively minimal variation as between the different types and brands of plastic bags used in the experiment.

TABLE 1
Peak Concentration of ClO₂ Gas

| | | Peak Dose |
|---|---|---|
| Brand Name | SKU/Lot Information | (ppm) |
| GREAT VALUE ® | 078742096650/340950 | 13 |
| (Walmart Apollo LLC) | | |
| ULINE ® (Uline Inc.) | N/A | 13 |
| HEFTY ® (Reynolds | 013700814068/OUR81406THG 737-883 | 13 |
| Consumer Products LLC) | | |
| SureFresh | 64156005225/843268 1905 | 15 |
| (SMG Brands Inc.) | | |
| GLAD ® | 012587790373/252753.001GC | 16 |
| (Glad Products Co.) | | |

### Example 3—Controlling ClO₂ Gas Release Profile

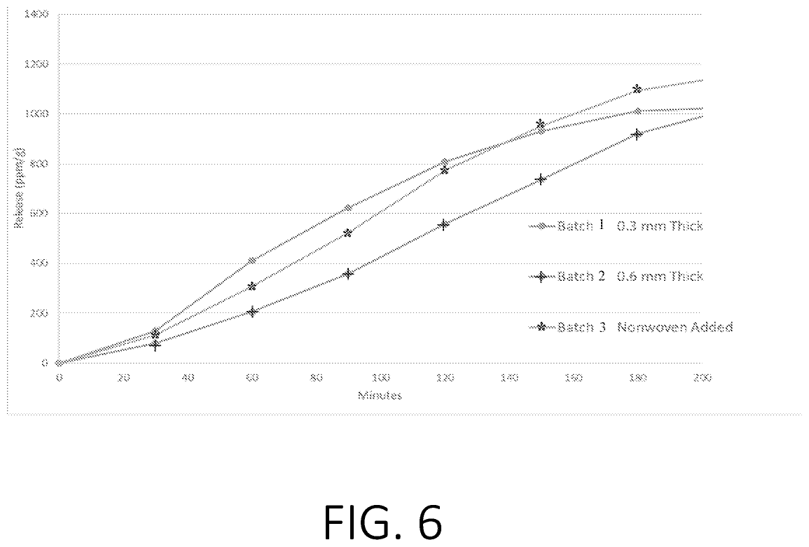

Another batch of polymer film was prepared according to the formulation and process set forth in Example 1. Three strip samples 10 mm×10 mm were prepared. Sample 1 (Batch 1) was 0.3 mm thick. Sample 2 (Batch 2) was 0.6 mm thick. Sample 3 (Batch 3) was less than 1 mm thick and nonwoven material was added during extrusion. Each strip of film was dipped into water and placed immediately into a sealable one gallon bag. The release rates of ClO₂ gas were measured. The results are set forth in FIG. 6. FIG. 6 demonstrates the capability of the system for control of the release rate of ClO₂ gas from the polymer film which can be designed by changing the thickness and mass of the film in the system.

### Example 4—Use of Marker as Chemical Indicator

A Sharpie™ Electric Pink #1927338 permanent marker was selected for use as a color indicator that would allow the user to vividly mark a line on both sides of the polymer film strip in accordance with the system herein. Polymer film according to Example 1 was prepared having a thickness of 0.3 mm and cut into strips of 10 mm×10 mm. The marker was used prior to wetting the strip and placing into the one gallon plastic resealable bag with an N95 respirator mask for disinfection. The marking was clearly visible on the strip on both sides prior to the disinfection cycle before activation with water and placement into the plastic bag. After the disinfection cycle, the lines on both sides of the film strip had disappeared and were no longer visible.

The disappearing of the marked line(s) qualitatively signified that the active strip had been activated during the bioburden reduction cycle in the plastic resealable bag. A disinfection cycle would be considered successful if (a) the indicating mark was observed to disappear completely from the strip within ten (10) hours from strip activation and (b) reappearance of the indicating mark was not observed for the duration of the experiment.

Repeated trial results indicated that the chemical indicator line generally disappeared by the second hour and did not reappear for 24 hours.

### Example 5—Use of Color Indicator to Show ClO₂ Activity

Polymer film was prepared as above, extruded and cut into 20 mm×75 mm×0.3 mm thick strips, each weighing approximately 0.499 g. The strips were placed and maintained in opaque plastic closed vials. A disinfection system according to an embodiment of the invention was prepared. An N95 respirator mask was placed into a one gallon plastic sealable bag. The strips were removed from the vial using tweezers. Each strip was again marked with a pink marker directly on both sides of the strip to function as a chemical indicator. Each strip was briefly submerged in water, one strip placed into the plastic bag, and the bag sealed with the sealable zipper. Care was taken to not expose the bags to direct sunlight. The water triggered a slow release of ClO₂ inside the plastic bag with the mask. The film strip was kept separate from the mask within the sealed bag, meaning that special care was taken to not place the respirator mask on the strip. As in Example 4, during the disinfection cycle, the pink indicator line on each strip disappeared substantially or completely on both sides of the film strip, qualitatively signifying that the strip had been activated for ClO₂ releasing activity. At the end of 10 hours, the cycle is complete and the bioburden reduction was measured.

### Example 6—Mask Disinfection

Polymer film strips according to Example 1 were prepared. A new, unused and clean N95 respirator mask was spot inoculated in four locations on the surface with Feline calicivirus, ATCC VR-782. Viral count was measured and recorded. The contaminated mask was placed into a one gallon sealable bag. Each film strip was completely dipped into water for 2-3 seconds, removed and placed into the sealable bag with the respirator mask. The bag was sealed for a set time to be disinfected according to the method herein. A second control sample was inoculated by identical procedure and placed into a separate one gallon sealable bag for the same amount of time. At the specified time, the bags were unsealed and the treated mask and the control mask were removed from their respective bags. The viral counts were measured on each mask. The mask that had been treated by the disinfection method herein showed a 5.2 log reduction (99.999%) in viral count as compared to the control sample.

### Example 7—Bacterial Efficacy Testing

Efficacy testing for bacterial count was performed. Testing was designed with a test strip weighing 0.499 g with a dosage as above with a qualitative chemical indicator as an internal process monitor and 3M™ 1860 N95 respirator masks (from the 3M Company, Minnesota, U.S.) for disinfection. Testing to measure bacterial count was performed pursuant to testing protocol of the U.S. FDA (Regulation PEUA200320). Efficacy testing was conducted on two gram-positive and two-gram negative bacteria and were tested at various dose sizes. The results of the bacteria testing in strip equivalents was converted into ppm-hours using a statistical population of ClO₂ ppm-hours generated by the test strip lot. The results are set forth in Table 2.

TABLE 2
Bacteria Test Results

| | | | | |
|---|---|---|---|---|
| Strip | Listeria | S. Aureus | Salmonella | E. coli |

| | |
|---|---|
| Equivalent | Avg. Log Reduction |

| | | | | |
|---|---|---|---|---|
| 2 | 2.45 | 2.95 | 3.14 | 3.88 |
| 5 | 4.49 | >6.13 | 4.61 | 4.94 |
| 8 | >6.28 | >6.13 | >6.29 | >6.49 |
| 12 | >6.28 | >6.13 | >6.29 | >6.49 |
| 15 | >6.28 | >6.13 | >6.29 | >6.49 |

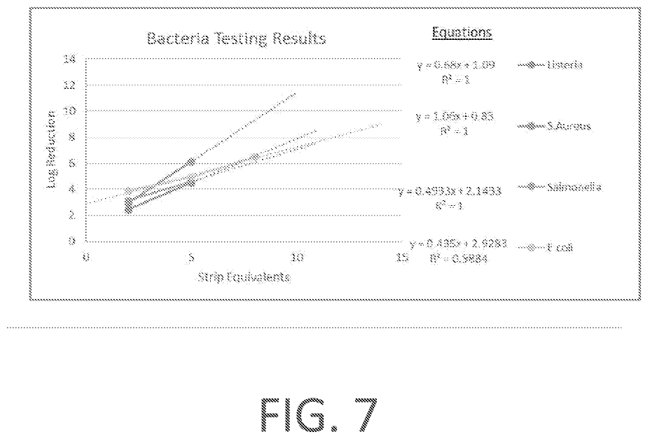

To define the exact dose required to achieve a minimum 3-log reduction in bacterial count, a mathematical curve was generated from the test results that defines the relationship between strip equivalent size (based on the dosage prepared in the Example) and the log reduction. It was determined that the dose required to achieve a minimum 3-log reduction on all four bacteria tested falls between a 2 and 5 strip equivalent dose. Based upon measurement and equations, Table 3 was generated to show the strip equivalent necessary to achieve a 3-log reduction for each type of bacteria and the results shown in FIG. 7.

TABLE 3
Calculated Strip Equivalent to Achieve a 3-log Reduction

| Bacteria | Log Reduction | Strip Equivalent |
|---|---|---|
| Listeria | 3.0 | 2.8 |
| S. Aureus | 3.0 | 2.0 |
| Salmonella | 3.0 | 1.8 |
| E. coli | 3.0 | 0.2 |

Based on the test results and calculations, the system and method can be controlled in account of the particular pathogen to be inhibited. For example, to address proliferation by Listeria, 2.8 strip equivalent is needed to achieve a minimum 3-log reduction. To disinfect a mask from S. aureus, two strip equivalents would be selected. And so forth.

### Example 8—Calculating ClO₂ Exposure

The actual exposure level of ClO₂ in ppm-hours generated by a polymer strip within a one gallon container equivalent system was calculated in order to quantify the exposure level of the chlorine dioxide to users during disinfection of an N95 respirator mask.

Measuring the exposure level in the actual bacteria test in a plastic bag was not possible because the method of measurement requires the headspace of the bag to be circulated through an external instrument, risking contamination of the instrument and a disruption of the treatment.

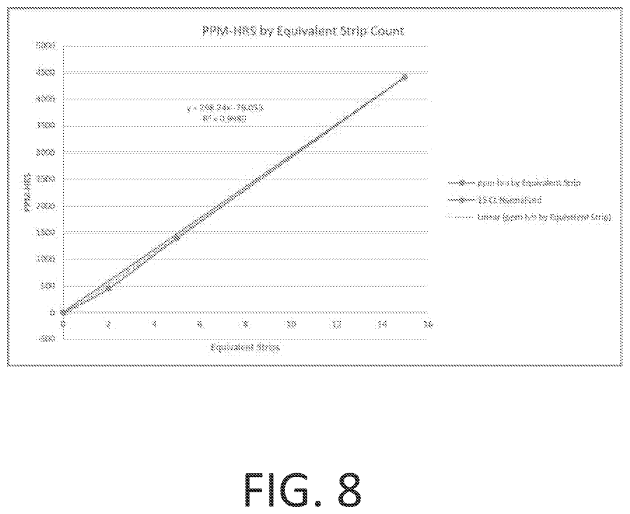

Thus, to calculate the Lower Specification Limit (LSL) for exposure, data for the system used in the bacteria testing was generated at a 2 strip, 5 strip, and 15 strip equivalent. The data confirmed the hypothesis that the exposure level is proportional to the strip size (in equivalent strips) within a given lot. To be conservative, it was assumed that the actual exposure level achieved in the bacteria testing was at the upper end of the distribution, so the LSL was set at Mean+3 Standard Deviations of the calculated 2.8 strip equivalent population. Based on the calculation, the LSL for ClO₂ exposure needed to ensure a minimum 3-log reduction of bacteria has been determined to be 1,163 ppm-hours FIG. 8 demonstrates the results of the measurement and calculation.

### Example 9—Viral Efficacy Testing—SARS-CoV-2

The SARS-CoV-2 (COVID 19) virus was tested at various dose sizes to determine efficacy of the current system and method herein. The virucidal testing was performed with a 3M™ 8511 N95 respirator. The test was repeated 5 times for each dosage.

The results of the virus testing in strip equivalents was converted into ppm-hours using a statistical population of ClO₂ ppm-hours generated by the test lot at ambient room temperature conditions and compared to an untreated N95 mask control. Efficacy testing was conducted at Microbac Laboratories on SARS-CoV using various doses of the polymer film prepared according to Example 1. The viral count results from the control and test masks are as shown in Table 4.

TABLE 4
SARS-CoV Test Viral Count Reduction Results

| Strip Count | Hours | Log Reduction |
|---|---|---|
| 1 | 2 | 1.25 |
| 3 | 2 | 3.68 |
| 5 | 4 | >5.18 |
| 10 | 4 | >5.18 |

To define the exact dose required to achieve a minimum 6-log reduction, a mathematical curve was generated that defines the relationship between strip equivalent size and log reduction. The graph for the SARS-CoV are set forth in FIG. 9.

It was calculated that it would require a 4.9 strip equivalent dose to achieve a minimum 6-log reduction of SARS-CoV-2. Using the same methodology as used for the bacteria results, the properties of a 4.9 strip population were calculated from the test data by rationing the test data from the actual strip size to a 4.9 strip equivalent size. To be conservative, it was assumed the actual exposure level in the SARS-CoV-2 testing was at the upper end of the distribution, so the Lower Specification Limit (LSL) was set at Mean+3 Standard Deviations of the calculated 4.9 strip equivalent population. The LSL for ClO₂ exposure required to ensure a minimum 6-log reduction on SARS-CoV was 2,035 ppm-hours with the 4.9 strip equivalent size.

Based on calculations, a final specification was developed to both achieve a minimum 3-log reduction of bacteria and a minimum 6-log reduction of SARS-CoV-2. A strip configuration of 15 strip equivalents was selected for the final device configuration to ensure robust bioburden reduction process capability.

### Example 10—Dissipation of ClO₂ Gas During Disinfection Cycle

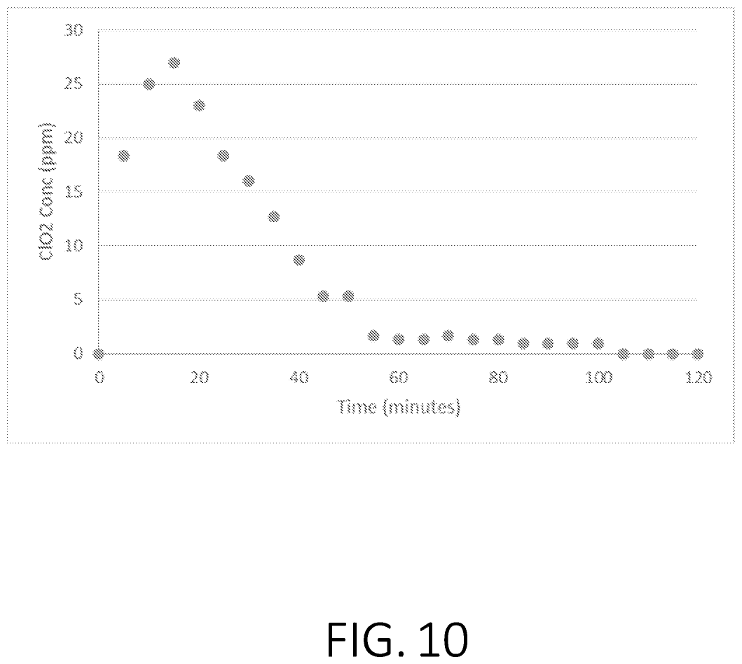

Samples of film were prepared as set forth in Example 1. A disinfection cycle was performed and monitored for 120 minutes and recorded. The results are set forth in FIG. 10. The experiment was repeated with and without an uncontaminated N95 mask within a sealed one gallon plastic bag. The concentration of ClO₂ gas was measured to be 15 ppm within 20 min of T₀. Within 120 min, the concentration of ClO₂ gas within the headspace of the sealed bag was measured to be substantially zero. This indicated that there remained no resulting or insubstantial amount of ClO₂ gas in the sealed bag, with no residual odor and no hazard for the user.

### Example 11—Controlling the ClO₂ Gas Release Profile

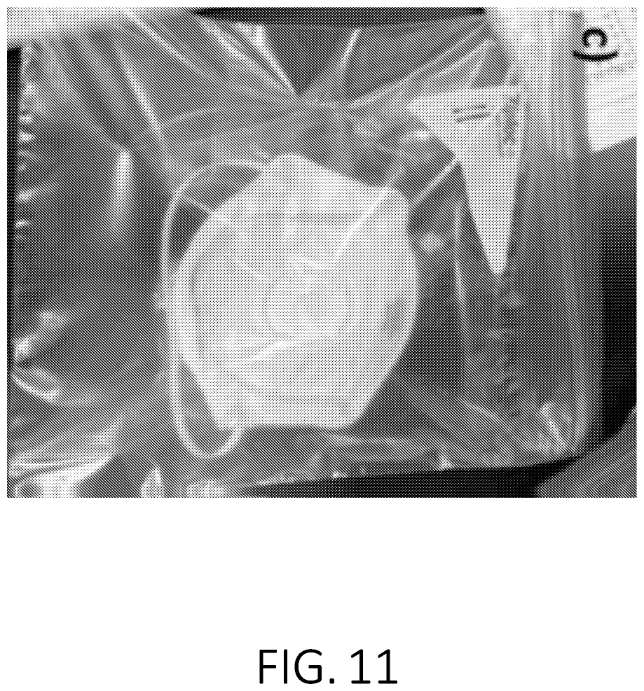

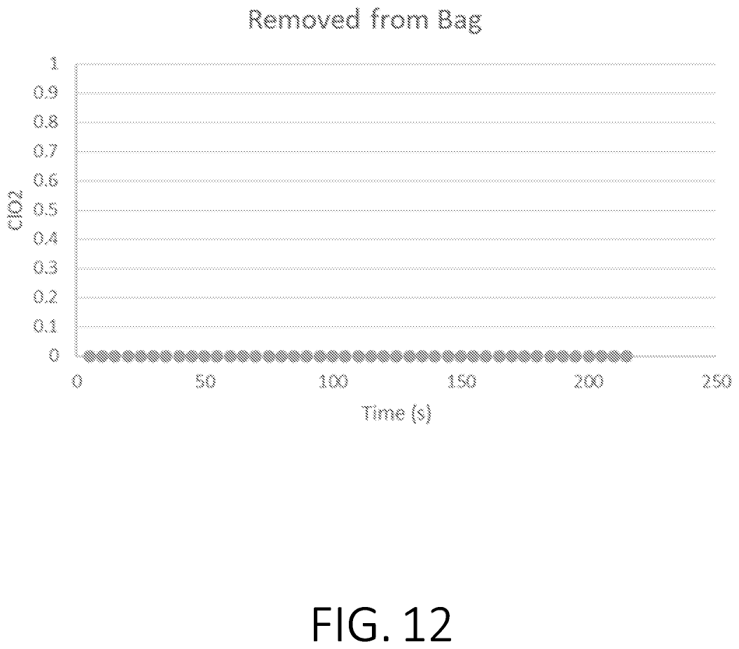

The experiment of Example 5 was repeated with uncontaminated N95 respirator masks placed into one gallon sealable plastic bags (one mask in each bag) with one strip of the activated antimicrobial film placed within the bags. A photograph of the system is shown in FIG. 11. The masks underwent a treatment of 30 and 120 minutes and removed from the bag. The masks were tested for any residual amount of ClO₂ on their surface with a Honeywell BW Solo ClO₂ Gas Detector (Honeywell International Inc., Charlotte, N.C., U.S.A.). It was measured that the level of ClO₂ gas on the masks immediately after removal from the sealed bag was zero, substantially zero or undetectable by the instrumentation used. The results are set forth in FIG. 12. This zero or substantially zero level of ClO₂ gas is considered as safe for use as set forth by CDC, FDA or EPA guidelines.

### Example 12—ClO₂ Gas Flow Test

The experiment of Example 5 was repeated, this time with two uncontaminated N95 respirator masks placed into one sealable plastic bag and tightly stacked one on top of the other within the bag. The masks were stacked so as to only allow a gas path (if at all) to flow through the mask(s). In order to test the chlorine dioxide activity effect on the masks, a piece of pH paper, purple in color (with a purple/white color change indicator element for ClO₂) was placed between the masks. One strip of the activated antimicrobial film was placed into the headspace of the bag. The bag was sealed for 30 minutes. A control sample of the pH paper (purple) was placed into a separate bag without any exposure to the antimicrobial polymer film. The bags were opened after 30 minutes and the pH film examined. The control strip remained purple indicating that no contact with ClO₂ had occurred. The pH paper from the system treated with the antimicrobial film was white, indicating contact with ClO₂. Since the pH test paper was placed in between the two masks, the results of the pH test demonstrated that the ClO₂ gas had permeated through one or both masks contacting the pH paper in order to turn it white from its original purple pH level. This result demonstrates that disinfection of the mask reaches beyond the surface and into and/or through the mask material.

### Example 13—Room Decontamination

The exposures to chlorine dioxide were modeled in this assessment for medical workers in a room where ClO₂ gas is released from the disinfection system herein containing N95 masks as set forth in Example 10, when the plastic bags are opened following a 30 minute treatment period. Two scenarios were modeled: (1) 10 treatment bags being opened in the room after 30 minutes; and (2) 100 treatment bags being opened in the room after a 30 minute treatment period. It was assumed that the bags would be opened at the rate of 5 bags per minute. Thus, for the 10-bag scenario, release of the chlorine dioxide occurred over a 2-minute period and for the 100-bag scenario, release occurred over a 20-minute period. In both cases, for modeling purposes the release rate was averaged over the release period. To maximize the 8-hour time-weighted average, the release period was modeled to occur at the beginning of a work shift.

### Example 14—Headspace Concentration with N95 Respirator in a Bag

A pharmacokinetics study was conducted to determine the chlorine dioxide gas concentration within the system in order to generate the Area-Under-the Curve (AUC), peak time Tₘₐₓ, and corresponding peak concentrations Cₘₐₓ with the N95 respirator in one gallon plastic bag.

One 15 strip equivalent dosage of polymer film of Example 1 was prepared. Using tweezers, the strip of film was completely submerged into tap water, and then immediately placed into the one gallon clear plastic sealable bag. A unused (uncontaminated) 3M™ 9211 N95 respirator was placed into the bag and bag was sealed. A continuous environmental monitoring system (ChlorDisys EMS™) was used to measure the chlorine dioxide gas concentration. The monitoring system had a lower detection limit accuracy of 40 ppm, therefore, the results reached non detectable measurements at 6 hours. An average of 30 trials were repeated.

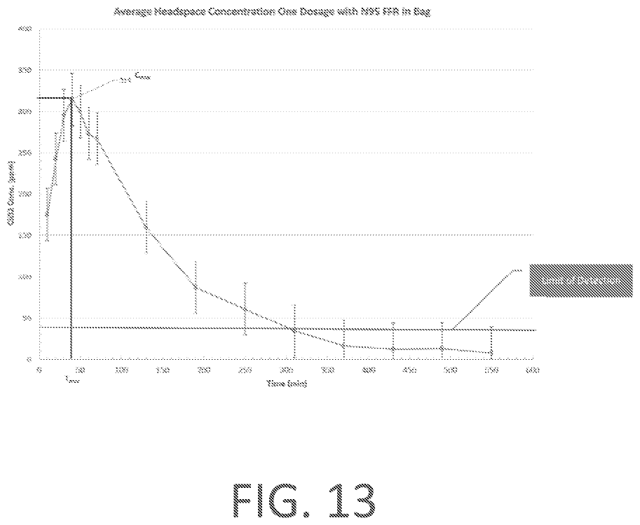

The trial measurements were recorded in FIG. 13. As shown, the Cₘₐₓ for the dosage in the test system was measured to be 314 ppm at a Tₘₐₓ of 40 minutes with the area under the curve (AUC) averaging 1,047 ppm-hours of total ClO₂ headspace concentration exposure. The ClO₂ headspace concentration levels for the system were thus found to be well above the 0.03-ppm limit indicated as safe by various health agencies for inactivating or mitigating flu-type viruses and bacteria.

### Example 15—Respirator Filtration Efficiency

Testing was conducted pursuant to NIOSH TEB-ABR-STP-0059 Initial Filtration Efficiency and Airflow Resistance Test Criteria [42 CFR § 84.180 (Airflow Resistance) and § 84.181 (Particle Efficiency)]. The tests were performed by SGS IBR Laboratories, a global leading inspection, verification, testing and certification company. The test was performed to determine non-powered air purifying particulate filter efficiency levels to ensure that the system herein meets the N95 rating requirement for N95 respirators.

3M™ 8511 N95 respirator masks were used with the antimicrobial chlorine dioxide entrained polymer film and all samples were subjected to 15 and 20 bioburden reduction cycles exposed to 15-strip and 20-strip equivalent of the dosage level (as set forth in Example 1). Results were recorded as shown in Table 5.

TABLE 5
SGS N95 Test Summary

| | Avg. | Avg. | | |
|---|---|---|---|---|
| | Inhalation | Exhalation | Avg. | Minimum |
| | Resistance | Resistance | Filtration | Filtration |
| Condition | [mm H₂O] | [mm H₂O] | Efficiency | Efficiency |
| Control (untreated) | 5.2 | 3.7 | 97.0% | 96.4% |
| (N = 6) | | | | |
| 15-strip equivalent dosage, | 5.3 | 4.5 | 97.0% | 95.2% |
| 15 repeated cycles | | | | |
| (N = 12) | | | | |
| 20-strip equivalent dosage, | 5.6 | 4.8 | 97.1% | 96.5% |
| 20 repeated cycles | | | | |
| (N = 12) | | | | |

The NIOSH 42 CFR 84.180 and 181 requirement acceptance criteria are as follows: Filtration Efficiency is ≥95%; Inhalation Resistance is ≤25 mm H₂O; and Exhalation Resistance is ≤35 mm H₂O. The results concluded that even when the N95 respirators were subjected to 20 repeated bioburden reduction cycles using 20-strip equivalent dosage, there was no difference between the control samples (untreated), with all test N95 respirators exceeding >97% FFR efficiency. Inhalation and exhalation resistance for 20 treatment cycles at 20-strip equivalent dosage was only 22.4% and 13.7% of the minimum requirement.

### Example 16—Fit Test Report

A further study was initiated to evaluate the effect of repeated bioburden reduction cycles of the present system on N95 respirator masks. Fit Testing was conducted in accordance with the Occupational Safety and Health Administration (OSHA) 1910.134 Standard from Title 29 CFR 1910.134 at Concentra Medical Center (5670 Fulton Industrial Blvd SW, Atlanta, Ga. 30336), which provides occupational health, urgent care, physical therapy, and wellness services.

Ten randomly selected test participants (three adult males and seven adult females) were provided N95 respirators to conduct the OSHA Respirator Fit Test Assessment. One N95 respirator type was tested (3M™ 8511 N95 mask). Five respirators had been subjected to 10 repeated bioburden reduction cycles and the remaining five respirators underwent 20 repeated cycles with the system of the invention, all at 20 strip equivalent dosage levels. Qualitative Fit Test (QLFT) protocol was utilized for this assessment, which is a Pass/Fail fit test to assess the adequacy of respirator fit that relies on an individual's response to the test agent.

The test generated a qualitative assessment record for the following parameters: (1) Condensation Nuclei Counter: ambient aerosol which required participant to wear the respirator for at least 5 minutes prior to assessment. A seal check was performed. (2) Assessment of comfort related to position of mask on nose, room for eye protection, adequate room to talk, and position of respirator on the face and cheeks. (3) Adequacy of fit was checked for proper chin placement, adequate strap tension, fit across nose bridge, proper size to span length from nose to chin, and tendency of respirator to slip. (4) Breathing was monitored for normal, deep, turning head left and right, and moving head up and down for one minute. (5) Talking was monitored. (6) Body movement was monitored for bending at the waist, jogging in place and normal breathing.

All of the respirators passed the qualitative requirements of the Fit Test. It was concluded that the system is suitable for use for as many as 20 repeated bioburden reduction cycles of N95 respirators with up to a 20 strip equivalent dosage levels.

### Example 17—Exposure to Healthcare Professionals

A Multi Chamber Concentration and Exposure Model (MCCEM) was administered by a 3rd party industrial hygienist certified D.A.B.T. (Diplomat of the American Board of Toxicology) of the Environmental Protection Agency (EPA). The MCCEM was peer reviewed by experts outside the EPA. MCCEM was developed under contract by Versar Inc. for the EPA Office of Pollution Prevention and Toxics, Economics, Exposure, and Technology Division, Exposure Assessment Branch (EAB). The model tested 100 masks disinfected by the method herein. Each mask was sealed in an individual one gallon plastic zipper sealable bag and underwent a decontamination cycle with the disinfection system herein. The masks were tested in a room half the size of the average hospital room. The sealed zipper bags were opened within 20 minutes after the disinfection cycle and the results recorded and calculated to be an estimated 8-hour Threshold Limit Value (TLV) level of 0.00138 ppm and a maximum airborne concentration of 0.16 ppm. The calculations concluded that the 8-hour concentration of chlorine dioxide gas is 1.38% of the Threshold Limit Value (TLV) of 0.1 ppm and 53% of the Short Term Exposure Limit (STEL) of 0.3 ppm set forth within the OSHA guidelines 1910.1000 (relating to air contaminants) for workers in any 8-hour work shift of a 40-hour work week. These studies showed that by use of the disinfection system and method herein, healthcare professionals can disinfect N95 and N95-equivalent masks in a safe and effective manner. Further, the system and method can be safely performed by healthcare workers in a room on-site at their medical facility.

### Example 18—Puncture Test

Tests were conducted to determine the ClO₂ concentration level a user may experience when a sealed bag is incidentally opened or may experience a hole while the bioburden reduction cycle attains peak concentration. The OSHA guidelines has set an 8-hour Threshold Limit Value (TLV) of 0.1 ppm for occupational exposures to chlorine dioxide in order to minimize the potential for respiratory tract irritation and bronchitis. The test acceptance criteria was set for results to be below 0.1 ppm for acceptable.

The following test criteria was followed. One 15-strip equivalent dosage of the polymer film of Example 1 was activated with water. A one gallon sealable bag was punctured with a pencil, (8 mm diameter pencil from Office Depot No. 2 HB), measuring appx. 8 mm diameter hole. Another one gallon sealable bag was punctured with a pen (0.5 mm diameter pen Pilot G2 roller ball 0.5 mm diameter point) forming an appx. 0.5 mm diameter hole. A third one gallon sealable plastic bag was left opened with mostly 100% of seal not intact at the top. Disinfection cycles were performed with 3M™ 1860 N95 respirators.

Each active strip was completely submerged via tweezers in water for several seconds, placed along with the N95 respirator into each one gallon plastic bag, respectively, and sealed. Each respirator underwent a bioburden reduction cycle to a Tₘₐₓ time of 30 minutes for peak concentration. The ClO₂ gas concentration was measured with a Honeywell BW Solo ClO₂ gas detector at one foot height above the bag. This detector has a standard measuring range for ClO₂ of 0-1 ppm with resolution of 0.01 ppm for an operating temperature of −20° C. to 40° C.

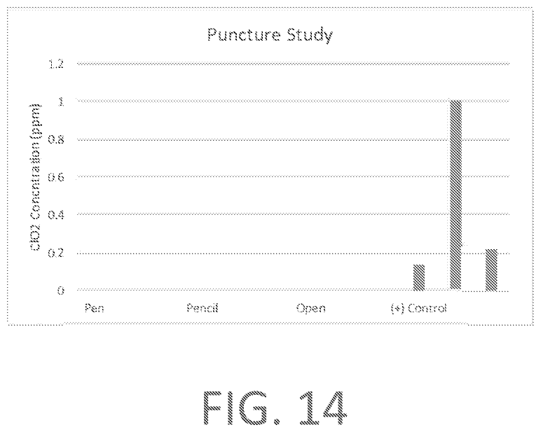

A control condition was set-up using seven (7) 15-strip equivalents per bag. Sealed bags were then punctured depending on condition tested (pen, pencil, open, control). A chemical indicator strip (LaMotte Chlorine Dioxide Indicator Strips) was placed into the plastic bag prior to sealing to verify that the film strips had been activated. The results were recorded in Table 6 and shown graphically in FIG. 14.

TABLE 6
ClO₂ Concentration Measured 1-Foot Height Above Bag

| | | ClO₂ level 1 ft height above bag |
|---|---|---|
| Bag Test Set-Up | Replicates | (ppm) |
| Positive Control | 3 | Up to 1 ppm |
| (1 for each set-up) | | |
| Open Bag | 3 | 0 |
| Pinhole 0.5 mm diameter | 3 | 0 |
| Hole 8 mm diameter | 3 | 0 |

It was found that at a one foot height directly above the one gallon sealed bag, there is no or an undetectable amount of ClO₂ measurement readings when the bag is open or has a small hole when the peak concentration is attained at 30 minutes. These results demonstrate an adequate degree of safety for the user during exposure to the system herein during a bioburden reduction process if the bag unseals or a hole is generated in the bag.

While the invention has been described in detail and with reference to specific examples, it will be apparent to one skilled in the art that various changes and modifications can be made therein without departing from the spirit and scope of the invention, thus the invention is further defined in scope by the following claims.

What is claimed is:

1. (canceled)

2. A method of disinfecting an object, the method comprising the following steps:
(a) placing the object to be disinfected into a container having an interior space therein, a headspace being formed of a portion of the interior space that is not occupied by the object;
(b) placing into the interior space a polymer composition comprising:
(i) a base polymer;
(ii) a chlorine dioxide gas forming agent; and
(iii) a channeling agent that forms channels though the base polymer;
(c) contacting the polymer composition with moisture to form chlorine dioxide gas; and
(d) enclosing the container sufficiently enough to allow the chlorine dioxide gas to accumulate in the headspace, wherein the chlorine dioxide gas disinfects the object;
wherein the amount of chlorine dioxide gas on the disinfected object is undetectable within 1 minute after removal.

3-5. (canceled)

6. A method of disinfecting an object, the method comprising the steps of:
(a) placing the object to be disinfected into a container having an interior space therein, a headspace being formed of a portion of the interior space that is not occupied by the object;
(b) placing into the interior space a polymer composition comprising:
(i) a base polymer;
(ii) a chlorine dioxide gas forming agent; and
(iii) a channeling agent that forms channels though the base polymer;
(c) contacting the polymer composition with moisture to form chlorine dioxide gas; and
(d) enclosing the container sufficiently enough to allow the chlorine dioxide gas to accumulate in the headspace, wherein the chlorine dioxide gas disinfects the object;
wherein the amount of chlorine dioxide gas in the ambient environment around the container is undetectable or considered Generally Recognized as Safe (GRAS) pursuant to Sections 201(s) and 409 of the United States Federal Food, Drug, and Cosmetic Act the entire time that the method is performed and immediately after removal of the object from the container.

7-11. (canceled)

12. The method of of claim 6, wherein the container is a polypropylene or polyethylene plastic resealable zipper storage bag having an interior volume of about one quart to about two gallons.

13. (canceled)

14. The method of of claim 6, wherein the object remains in the sealed container for a period of about 10 minutes to about 10 hours.

15. (canceled)

16. (canceled)

17. The method of of claim 14, wherein the object remains in the sealed container for a period of about 10 minutes to about 2 hours.

18. (canceled)

19. (canceled)

20. The method of of claim 6, wherein the polymer composition does not physically contact the object within the sealed container.

21. (canceled)

22. A method of disinfecting an N95 respirator mask, the method comprising the steps of:
(a) placing at least one N95 respirator mask into a sealable container, a headspace being formed of a portion of the interior space of the container that is not occupied by the mask;
(b) placing into the container a polymer composition comprising:
(i) a base polymer;
(ii) a chlorine dioxide gas forming agent comprising a chlorite salt; and
(iii) a channeling agent that forms channels though the base polymer;
(c) contacting the polymer composition with moisture in liquid form to form chlorine dioxide gas; and
(d) closing the container completely or sufficiently enough to allow the chlorine dioxide gas to accumulate in the headspace, wherein the chlorine dioxide gas permeates through the mask and disinfects the mask;
wherein the mask retains at least 95% of its filtration efficacy after 10 cycles of disinfection.

23-26. (canceled)

27. The method of claim 22, wherein only a single mask is provided in the container at a time.

28. (canceled)

29. (canceled)

30. The method of claim 22, wherein elastomer straps or bands are coupled to the mask and the amount of stretch performance of the elastomer straps or bands is not substantially changed after up to 10 cycles of disinfection.

31-35. (canceled)

36. The method of claim 6, wherein the moisture is water, acetone or an alcohol.

37. (canceled)

38. The method of claim 6, wherein the polymer composition is contacted with moisture in liquid form by submerging or dipping the polymer composition therein.

39. The method of claim 6, wherein the polymer composition is contacted with moisture in liquid form by pouring, spraying or spritzing the moisture onto the polymer composition or by contacting the polymer composition with a solid surface that comprises moisture thereon.

40-43. (canceled)

44. The method of claim 6, wherein the polymer composition is contacted with the moisture within the container after the container is closed or substantially closed.

45-49. (canceled)

50. The method of claim 6, wherein the concentration of the chlorine dioxide gas forming agent in the polymer composition is about 50% by weight of the total weight of the polymer composition and wherein the concentration of the channeling agent in the polymer composition is in a range of from 2% to 15% by weight of the total weight of the polymer composition.

51. (canceled)

52. The method of claim 6, wherein the distribution of the chlorine dioxide gas forming agent within the polymer composition is essentially homogeneous.

53. (canceled)

54. (canceled)

55. The method of claim 6, wherein the polymer composition comprises silica or silica gel.

56-59. (canceled)

60. The method of claim 6, wherein the chlorine dioxide gas forming agent consists essentially of from 10% to 15% sodium chlorite, from 5% to 15% calcium chloride, and from 70% to 80% silica gel by weight based on the total weight of the chlorine dioxide gas forming agent, wherein the chlorine dioxide gas forming agent and/or the silica gel has a pH of from 1.4 to 3.1.

61. The method of claim 6, wherein the polymer composition comprises sodium chlorite, calcium chloride, silica gel, ethyl vinyl acetate and polyethylene glycol.

62. (canceled)

63. The method of claim 6, wherein the polymer composition further comprises a color indicator to indicate that chlorine dioxide gas has been formed, wherein the color or shade of at least a portion of the polymer composition prior to contact with moisture is different than the color or shade of the at least a portion of the polymer composition after formation of the chlorine dioxide gas.

64. The method of claim 6, further comprising providing a chlorine dioxide gas indicator into the container to measure the concentration of chlorine dioxide gas inside the container or to otherwise indicate that chlorine dioxide gas has been formed.

65-67. (canceled)

68. The method of claim 6, wherein the polymer composition and/or the chlorine dioxide gas forming agent is coated with at least one extended release coating or layer to effectuate a pre-determined release profile of the chlorine dioxide gas within the closed container.

69. (canceled)

70. The method of claim 6, wherein a peak concentration of chlorine dioxide gas in the container is reached after contact with moisture in a period of from 10 minutes to 2 hours.

71. The method of claim 6, wherein the concentration of chlorine dioxide gas formed in the container effectuates a reduction of at least one type of infectious agent contained on the object to be disinfected, the reduction being at least a 1 log base 10 reduction in colony forming units per gram (CFU/g) compared to the initial number of colony forming units of the at least one type of infectious agent at ambient temperature.

72-75. (canceled)

76. The method of claim 71, wherein the infectious agent is norovirus.

77-79. (canceled)

80. The method of claim 6, wherein the level of disinfection renders the object sterilized pursuant to guidelines for sterilization set forth by the United States Centers for Disease Control pursuant to the Code of Federal Regulations, Title 21, Section 110.3(o).

81. (canceled)

82. The method of claim 80, wherein the object to be disinfected is a medical device.

83. The method of claim 82, wherein the medical device is an endoscope.

84-89. (canceled)

90. The method of claim 6 for use in a package for the distribution and/or storage of a product.

91. The method of claim 6, wherein the moisture is applied to the polymer composition via a wet roller mechanism during an in-line packaging process.

92. The method of claim 6, wherein the container is a sealable chamber with an openable door that is operable to be locked shut to seal the object within the chamber during disinfection.

93. (canceled)

94. The method of claim 92, comprising a chlorine dioxide gas sensor within the chamber configured to detect chlorine dioxide gas concentration within the chamber, the sensor being configured to transmit a signal indicative of the chlorine dioxide gas concentration within the chamber at a given time to a readout display, wherein the door is configured not to unlock until detected concentration of chlorine dioxide within the chamber has reached a predetermined safe level for human exposure.

95-97. (canceled)

98. A method of disinfecting a room, the method comprising the steps of:
(a) placing into the room a polymer composition comprising:
(i) a base polymer;
(ii) a chlorine dioxide gas forming agent; and
(iii) a channeling agent that forms channels though the base polymer;
(b) contacting the polymer composition with moisture to form chlorine dioxide gas; and
(c) substantially sealing the room to allow the chlorine dioxide gas formed by the polymer composition to accumulate in the room, wherein the chlorine dioxide gas disinfects the room, including air and objects within the room.

99. (canceled)

100. (canceled)

101. The method of claim 98, wherein the disinfection is performed at a safe level while one or more people occupy the room.

102-114. (canceled)

115. The method of claim 22, wherein the container is a polypropylene or polyethylene plastic resealable zipper storage bag that is a quart to two gallons in volume.

116. The method of claim 71, wherein the infectious agent is hepatitis virus.
